(19) United States
(12) Patent Application Publication
(10) Pub. No.: US 2009/0035740 A1
(43) Pub. Date: Feb. 5, 2009

(54) SYSTEMS AND METHODS FOR REMOTE CONTROLLED INTERACTIVE TRAINING AND CERTIFICATION

(75) Inventors: Jaime C. Reed, Mukwonago, WI (US); Samuel K. Foos, Big Bend, WI (US); Anthony S. Fischer, Mukwonago, WI (US); Thomas J. Monfre, Menomonee Falls, WI (US); Todd R. Nelson, Rochester, WI (US)

Correspondence Address: Casimir Jones, S.C., 440 Science Drive, Suite 203, Madison, WI, 53711, US

(73) Assignee: MONSTER MEDIC, INC., Mukwonago, WI (US)

(21) Appl. No.: 12/182,604

(22) Filed: Jul. 30, 2008

Related U.S. Application Data
(60) Provisional application No. 60/962,596, filed on Jul. 30, 2007.

(51) Int. Cl. G09B 23/28; A61N 1/00

(52) U.S. Cl. 434/265; 607/5

(57) ABSTRACT

The present invention relates to systems and methods for training, testing and/or certifying one or more subjects (e.g., at the same time (e.g., in the use of an automated external defibrillator (AED) and/or cardiopulmonary resuscitation (CPR))). In particular, the present invention relates to remote bi-directional communications (e.g., data delivery, acquisition, and/or maintenance). For example, the present invention provides communication between a handheld AED simulation device (e.g., comprising a processor) and one or more simulation devices (e.g., a mannequin (e.g., comprising a processor and/or one or more sensors) connected thereto and/or components (e.g., controls and/or sensors) associated therewith. The present invention also provides a system configured to relay information (e.g., instructions and/or performance data) between one or more processors (e.g., housed on a personal computer or other device) and one or more additional processors (e.g., housed on a simulation device (e.g., a mannequin comprising one or more sensors (e.g., hall effect sensors and/or switch sensors associated therewith).

This application claims priority to U.S. Provisional Patent Application Ser. No. 60/962,596 filed Jul. 30, 2007, hereby incorporated by reference in its entirety.

### FIELD OF THE INVENTION

The present invention relates to systems and methods for training, testing and/or certifying one or more subjects (e.g., at the same time). In particular, the present invention relates to remote bi-directional communications (e.g., data delivery and/or acquisition, maintenance, and/or control) between a server (e.g., available on the internet) and one or more personal computers and/or simulation devices (e.g., a mannequin comprising one or more sensors) connected thereto and/or components (e.g., controls and/or sensors) associated therewith.

### BACKGROUND OF THE INVENTION

Sudden cardiac arrest (SCA) is a leading cause of death in the United States and Canada. Although estimates of the annual number of deaths due to out-of-hospital SCA vary widely, data for the Centers for Disease Control and prevention estimates that in the United States approximately 330,000 people die annually in the out-of-hospital and emergency department settings from coronary heart disease. About 250,000 of these deaths occur in the out-of-hospital setting.

In general, victims of SCA demonstrate ventricular fibrillation at some point in their arrest. Resuscitation is most successful if defibrillation is performed in about the first 5 minutes after collapse. Because the time frame from a call to 911 and arrival of EMT and/or paramedics to a SCA victim is typically longer than 5 minutes, achieving high survival rates depends on a public trained in cardiac pulmonary resuscitation (CPR) and/or automated external defibrillator (AED) usage.

Accordingly, there exists a need for well organized public access to training, testing and/or certification in these and other immediate care procedures.

### SUMMARY OF THE INVENTION

The present invention relates to systems and methods for training, testing and/or certifying one or more subjects (e.g., at the same time (e.g., in the use of an automated external defibrillator (AED) and/or cardiopulmonary resuscitation (CPR))). In particular, the present invention relates to remote bi-directional communications (e.g., data delivery, acquisition, and/or maintenance). For example, the present invention provides communication between a handheld AED simulation device (e.g., comprising a processor) and one or more simulation devices (e.g., a mannequin (e.g., comprising a processor and/or one or more sensors) connected thereto and/or components (e.g., controls and/or sensors) associated therewith. The present invention also provides a system configured to relay information (e.g., instructions and/or performance data) between one or more processors (e.g., housed on a personal computer or other device) and one or more additional processors (e.g., housed on a simulation device (e.g., a mannequin comprising one or more sensors (e.g., hall effect sensors and/or switch sensors associated therewith).

Accordingly, in some embodiments, the present invention provides an interactive cardiac pulmonary resuscitation (CPR) and/or automated external defibrillator (AED) training and/or testing system comprising: an AED simulation device (e.g., wherein the AED device comprises a microcontroller; a mannequin (e.g., wherein the mannequin comprises a microcontroller); and a bi-directional communication link between the AED simulation device and the mannequin (e.g., between the AED simulation device microcontroller and the mannequin microcontroller). In some embodiments, the AED simulation device comprises firmware. The present invention is not limited to any particular type of firmware. Indeed, a variety of different types of firmware are utilized including, but not limited to, universal serial bus (USB) firmware, audio firmware, and microprocessor firmware. In some embodiments, the AED simulation device comprises a USB drive and/or controller. In some embodiments, the USB drive is utilized to update firmware of the AED simulation device. In some embodiments, testing and/or training data is stored on a USB drive connected to the AED simulation device. In some embodiments, user performance data is stored on a USB drive connected to the AED simulation device. In some embodiments, data stored on a USB drive (e.g., user performance data, testing and/or training data, system operation data, or other type of data described herein) is uploaded to an accessible server (e.g., Web server (e.g., via connecting the USB drive to a PC with internet access)) that collects, manages and/or processes the data. In some embodiments, an accessible server (e.g., Web server) is used to manage, troubleshoot, update and/or otherwise alter operational and functional parameters an AED simulation device, (e.g., via downloading information to a USB drive and inserting the drive into the AED simulation device) and/or simulation system connected thereto. Thus, in some embodiments, the present invention provides a system comprising a server (e.g., Web server) that collects, manages and/or processes information (e.g., user performance data located on a USB drive (e.g., of an AED simulation device described herein). In some embodiments, information is communicated between the AED simulation device microcontroller, the mannequin microcontroller, and the USB drive. In some embodiments, testing and/or training data comprises user performance data. In some embodiments, the bi-directional communication link comprises a USB cable. However, the present invention is not so limited. Indeed, any bi-directional communication means can be utilized for communicating and/or connecting between the AED simulation device microcontroller and the mannequin microcontroller including, but not limited to, cable (e.g., USB type I, USB type 2, USB type 3, serial cable, etc.), infrared, BLUETOOTH, WIFI (e.g., IEEE 802.11a, b, g, p, etc), WIMAX, ZIGBEE (e.g., ZIGBEE wireless networking tools), or other type of technology. In some embodiments, the AED simulation device and the mannequin comprise one or more sensors. The present invention is not limited by the type of sensor utilized nor by the location of the sensor. A variety of different types of sensors may be utilized including, but not limited to, hall effect sensors, tactile switch sensors, magnetic proximity sensors or other type of sensor described herein. The present invention is not limited by the information and/or location of the one or more sensors. Indeed, one or more sensors may be utilized to monitor and/or record head tilt, pulse check, compression and/or rise, ventilation, location (e.g., AED pad placement), force (e.g., a user pushing on an on/off button or a charge button) or other type of user performance criteria. In some embodiments, the sensors collect user performance data (e.g., that is stored on a USB drive). In some embodiments, a system of the present invention comprises a guide system (e.g., that assists a user in training with a system of the present invention). The present invention is not limited by the type of guide system. Indeed, a variety of guide systems may be utilized including, but not limited to, a voice prompt guide system, a train to light guide system, a brail guide system, and/or any combination thereof. In some embodiments, the train to light guide system comprises a protocol that places a light indicator at a location where a user (e.g., a trainee learning CPR and/or AED usage with a system of the invention) needs to perform an action (e.g., of CPR and/or AED usage). In some embodiments, a light is illuminated (e.g., at a site where a user needs to perform an action) and extinguished upon proper completion of an action. In some embodiments, the present invention provides a brail guide system (e.g., CPR and/or AED training and/or testing system) comprising brail prompts. In some embodiments, the AED simulation device and/or the mannequin comprise one or more light-emitting diodes (LEDs). In some embodiments, the one or more LEDs are located at one or more positions comprising a shock switch of the AED device, an on/off button of the AED device, location of chest rise and/or compression of the mannequin, location of AED pad placement on the mannequin, location of head tilt on the mannequin, location of pulse check on the mannequin, and/or location of nose pinch of the mannequin. In some embodiments, firmware present in the AED simulation device utilizes a protocol that illuminates the one or more LEDs at a location where a user of the system needs to perform an action and extinguishes the one or more LEDs when an action has been properly completed. In some embodiments, the mannequin comprises a translucent cover (e.g., skin). The present invention is not limited by the type of translucent material utilized as a cover. Indeed, a variety of materials may be utilized including, but not limited to, poly vinyl chloride or other type of translucent material. In some embodiments, the AED simulation device comprises a speaker. In some embodiments, the speaker provides voice prompts to a user of the system. In some embodiments, the voice prompts comprise instructions to a user training on the system in the performance of CPR and/or AED usage. In some embodiments, the system comprises an accessible (e.g., internet accessible) server. In some embodiments, the server houses software for training, testing and/or certifying a user in CPR and/or AED usage. In some embodiments, the server houses a database of user performance data. In some embodiments, the database is utilized to generate performance statistics (e.g., trend data). In some embodiments, the system comprises a website. In some embodiments, the website provides a user interface that allows a user (e.g., an administrator) access to user performance data, certification information, or trending data.

The present invention also provides an interactive cardiac pulmonary resuscitation (CPR) and/or automated external defibrillator (AED) training and/or testing system comprising: an AED training unit assembly comprising a microcontroller; a shock pad assembly (e.g., comprising a pair of defibrillator pads, a pair a wires, and an attachment); and a mannequin.

The present invention also provides a mannequin. In some embodiments, the mannequin comprises a head and an inner shell assembly comprising built in ramps and a plurality of rail surfaces on the shell for the head to saddle and rotate upon. In some embodiments, a head mount bolt mounts the head to the inner shell assembly. In some embodiments, the head mount bolt rides on a channel of the inner shell assembly that is an arc. In some embodiments, pivoting the head on the arc channel simulates the movement of a human head in the motion of lifting the chin and tilting the head back to open a human's airway.

The present invention also provides a method of training and/or testing in the methodologies of CPR and/or AED usage comprising providing a system of the invention, and a user (e.g., trainee and/or test taker), and providing the user a USB (e.g., thumb) drive for use with the system. In some embodiments, the USB drive is configured (e.g., by an administrator/instructor) prior to use by a trainee and/or test taker. In some embodiments, the administrator/instructor connects the thumb drive to a computer with internet connection and logs onto a website configured to provide test and/or training information to the USB drive. In some embodiments, once connected to the website, the administrator/instructor is required to register (e.g., with a user name and/or password). In some embodiments, once logged in, the website prepares the USB drive (e.g., after the administrator/instructor requests the website to prepare the USB drive (e.g., by pressing the appropriate button/link on the website)). In some embodiments, once requested, website software searches for a drive with a volume of a particular label. In some embodiments, if the website finds the volume on the USB drive, the website identifies the drive with the particular label as the USB drive. In some embodiments, the website identifies the presence or absence of a test file on the USB drive (e.g., by name of the test file on the USB drive). In some embodiments, if there is no test file identified, then the USB drive is erased and a test file (e.g., with a test file name) is loaded onto the USB drive. In some embodiments, if there is a test file already on the USB drive (e.g., with a test file label) then the test file is opened for reading. In some embodiments, the test file contains test information including, but not limited to, test identification number, date, time, user performance data, etc.). In some embodiments, one or more types of written tests are administered together with (e.g., before, during and/or after) training and/or testing with a device of the present invention. In some embodiments, correlating test identification number (e.g., on a written test and/or training and/or testing using a device of the present invention (e.g., utilizing a USB drive and corresponding drive filename to provide and/or store data (e.g., user performance data)) is utilized to generate a database, calculate statistics (e.g., trending data), improve performance, prevent cheating, etc.

### DESCRIPTION OF THE DRAWINGS

FIG. 1 shows a diagram of one embodiment of a system of the present invention wherein an accessible server interacts remotely with a plurality of personal computers and/or simulation devices connected thereto.

FIG. 2 shows a diagram of a system comprising software components and an accessible server in one embodiment of the present invention.

FIG. 3 shows a diagram depicting various locations of mannequin and AED sensors in one embodiment of the invention.

FIG. 4 shows a diagram depicting a data acquisition module and channel definitions in one embodiment of the present invention.

FIG. 5 shows an example of CPR and/or AED testing flow logic in one embodiment of the present invention.

FIG. 6 shows an example of CPR and/or AED testing flow logic in one embodiment of the present invention.

FIG. 7 shows one embodiment of skills testing provided by systems and methods of the invention.

FIG. 8 shows a mannequin and its component parts in one embodiment of the present invention.

FIG. 9 shows a diagram of an embodiment of an AED device of the present invention.

FIG. 10 shows a diagram of a system in one embodiment of the invention comprising an AED device comprising a microprocessor in communication with a mannequin comprising a microprocessor and subcomponents in communication with both the AED microprocessor and the mannequin microprocessor.

FIG. 11 shows a diagram of a system in one embodiment of the invention comprising an AED device in communication with a mannequin, wherein both the mannequin and the AED device comprise sensors utilized to collect information regarding a subjects performance.

FIG. 12 shows a diagram of a system in one embodiment of the invention comprising an AED device and a mannequin, wherein both the mannequin and the AED device comprise light-emitting diodes (LEDs) utilized to assist a subject during training procedures utilizing the system.

FIG. 13 shows a diagram of a mannequin and the location of LEDs that can be illuminated, for example, during a training session, in one embodiment of the invention.

FIG. 14 shows a diagram of a system in one embodiment of the invention comprising an AED device, a mannequin, and means for communication between the two, as well as other components of the system.

FIG. 15 shows a diagram of a system ready for use in one embodiment of the invention comprising an AED device in communication with a mannequin.

FIG. 16 shows a chart depicting an algorithm utilized for testing and/or training utilizing a system in one embodiment of the invention.

FIG. 17 shows how a head component of a mannequin attaches to an inner shell assembly of a mannequin in one embodiment of the invention.

### DEFINITIONS

To facilitate an understanding of the present invention, a number of terms and phrases are defined below:

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein the term “encode” refers to the process of converting one type of information or signal into a different type of information or signal to, for example, facilitate the transmission and/or interpretability of the information or signal. For example, audio sound waves can be converted into (i.e., encoded into) electrical or digital information. Likewise, light patterns can be converted into electrical or digital information that provides and encoded video capture of the light patterns.

As used herein, the term “client-server” refers to a model of interaction in a distributed system in which a program at one site sends a request to a program at another site and waits for a response. The requesting program is called the “client,” and the program that responds to the request is called the “server.” In the context of the World Wide Web, the client is a “Web browser” (or simply “browser”) that runs on a computer of a user; the program which responds to browser requests by serving Web pages is commonly referred to as a “Web server.”

As used herein, the term “Internet” refers to any collection of networks using standard protocols. For example, the term includes a collection of interconnected (public and/or private) networks that are linked together by a set of standard protocols (such as TCP/IP, HTTP, and FTP) to form a global, distributed network. While this term is intended to refer to what is now commonly known as the Internet, it is also intended to encompass variations that may be made in the future, including changes and additions to existing standard protocols or integration with other media (e.g., television, radio, etc). The term is also intended to encompass non-public networks such as private (e.g., corporate) Intranets.

As used herein the term “security protocol” refers to an electronic security system (e.g., hardware and/or software) to limit access to processor to specific users authorized to access the processor. For example, a security protocol may comprise a software program that locks out one or more functions of a processor until an appropriate password is entered.

As used herein the term “resource manager” refers to a system that optimizes the performance of a processor or another system. For example a resource manager may be configured to monitor the performance of a processor or software application and manage data and processor allocation, perform component failure recoveries, optimize the receipt and transmission of data, and the like. In some embodiments, the resource manager comprises a software program provided on a computer system of the present invention.

As used herein the term “in electronic communication” refers to electrical devices (e.g., computers, processors, communications equipment, etc.) that are configured to communicate with one another through direct or indirect signaling. For example, a computer configured to transmit (e.g., through cables, wires, infrared signals, telephone lines, satellite, etc) information to another computer or device, is in electronic communication with the other computer or device.

As used herein the term “transmitting” refers to the movement of information (e.g., data) from one location to another (e.g., from one device to another) using any suitable means.

### DETAILED DESCRIPTION OF THE INVENTION

The present invention relates to systems and methods for training, testing and/or certifying one or more subjects (e.g., at the same time (e.g., in the use of an automated external defibrillator (AED) and/or cardiopulmonary resuscitation (CPR))). In particular, the present invention relates to remote bi-directional communications (e.g., data delivery, acquisition, and/or maintenance). For example, the present invention provides communication between a handheld AED simulation device (e.g., comprising a processor) and one or more simulation devices (e.g., a mannequin (e.g., comprising a processor and/or one or more sensors) connected thereto and/or components (e.g., controls and/or sensors) associated therewith. The present invention also provides a system configured to relay information (e.g., instructions and/or performance data) between one or more processors (e.g., housed on a personal computer or other device) and one or more additional processors (e.g., housed on a simulation device (e.g., a mannequin comprising one or more sensors (e.g., hall effect sensors and/or switch sensors associated therewith).

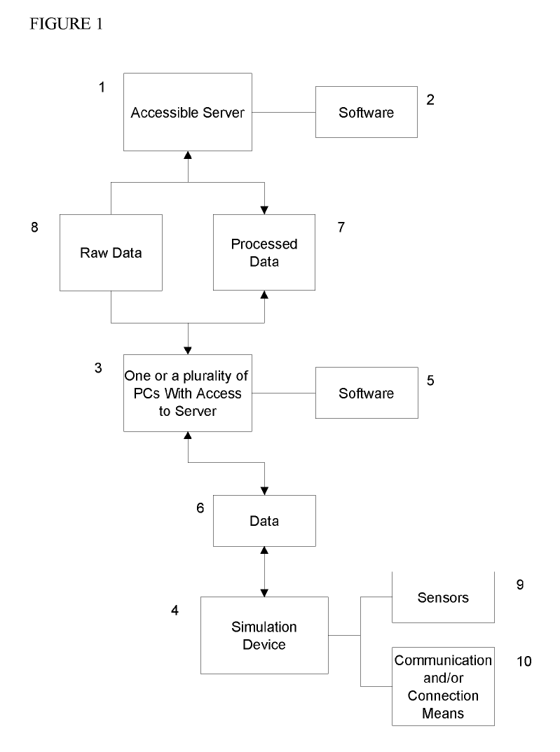

Accordingly, in some embodiments, the present invention provides a system as depicted in FIG. 1. In FIG. 1, a server 1 (e.g., connected to the internet) housing software 2 (e.g., a training program, a testing program, and/or data processing program (e.g. for determining user performance (e.g., identifying users that achieve or fail performance criteria for certification))) is accessible (e.g., via the internet) by one or a plurality of personal computers 3 (PC) that is connected to a simulation device 4 (e.g., a mannequin). In some embodiments, the PC 3 comprises software 5 (e.g., mannequin driver software and/or communication software (e.g., for sending and/or receiving information to and/or from the mannequin 4 and/or server 1)). In some embodiments, data 6 is acquired from the simulation device 4 by software 5 housed on the PC 3. In some embodiments, software 5 housed on the PC 3 permits the server 1 to download data 7 (e.g., testing data (e.g., a testing program), training data (e.g., a training program), performance data, certification data, etc.) from the server 1 to the PC 3 and/or simulation device 4. In some embodiments, software 5 housed on the PC 3 uploads raw data 8 (e.g., un-processed data) from the simulation device 4 onto the server 1. In some embodiments, software 5 on the PC 3 processes data 6 acquired from the simulation device 4 prior to uploading the processed data 7 onto the server 1. The present invention is not limited by the type of data 6 acquired from the simulation device 4. Indeed, a variety of types of data 6 can be acquired including, but not limited to, data obtained from one or more sensors 9 present on or within the simulation device 4 (e.g., head tilt sensors, pulse sensors, compression sensors, ventilation sensors, location sensors (e.g., for an AED device), charge sensors, etc.) user performance data, and sensor status data. Similarly, the present invention is not limited by the type of data 6 sent from the PC 3 to the simulation device 4. Indeed, a variety of types of data 6 can be sent from the PC 3 to the simulation device 4 including, but not limited to, real-time instructions from the server (e.g., related to simulation device activity). In some embodiments, a simulation device 4 comprises means 10 for communicating and/or connecting with a PC 3. The present invention is not limited by the means 10 for communicating and/or connecting. Indeed, a variety of different means 10 for communicating and/or connecting are contemplated including, but not limited to, via cable (e.g., USB type I, USB type 2, USB type 3, serial cable, etc.) and/or wireless (e.g., infrared, BLUETOOTH, WIFI (e.g., IEEE 802.11a, b, g, p, etc), WIMAX, ZIGBEE (e.g., ZIGBEE wireless networking tools), etc.) technologies.

In some embodiments, the present invention provides real-time access for a PC 3 connected to a simulation device 4 to a remote server 1 (e.g., Web server) via a communication network (e.g., the Internet). In some embodiments, the server 1 (e.g., that houses software 2 for training, testing and/or certifying a user) enjoys bi-directional real-time data (e.g., sound, electrical signals sent to and/or acquired from sensors 9 attached to simulator device 4) communications with the PC 3 connected to a simulation device 4 via a broad range of communication link systems. The PC 3 connected to a simulation device 4 is in turn in communication (e.g., via the Internet, telemetric communication, etc.) with the server 1 such that the PC 3 connected to a simulation device 4 may uplink to the server 1 or the sever 1 may down link to the PC 3 connected to a simulation device 4, as needed.

In some embodiments, components (e.g., software, firmware, an embedded component and/or type of functional component) of the PC 3 and/or simulation device 4 connected thereto are remotely maintained (e.g., updated, configured, assessed, debugged, evaluated to ensure proper functioning and/or performance, etc.) by down linking software, firmware and/or other components from the server 1 (e.g., Web server). In some embodiments, the PC 3 and/or simulation device 4 connected thereto is remotely monitored (e.g., by software 2 configured to monitor), assessed and/or upgraded as needed (e.g., by importing information and/or instructions via the internet (e.g., from a server) or other source of information (e.g., CD, DVD, flash card, memory stick, or other source of date storage)). In some embodiments, operational and/or functional software 5 run by the PC 3 may be remotely adjusted, upgraded or changed. For example, in some embodiments, software 2 run by the server 1 may download to a remote PC 3 and/or simulation device 4 connected thereto information that upgrades the simulation device (e.g., mannequin) firmware and/or simulation device 4 driver software run on the PC 3 and/or simulation device.

In some embodiments, the invention provides a communications scheme that provides an integrated and efficient method and/or structure of information management in which various networks such as Community access Television (CAT), Local area Network (LAN), a wide area network (WAN) Integrated Services Digital Network (ISDN), the Public Switched telephone Network (PSTN), the Internet, a wireless network, an asynchronous transfer mode (ATM) network, a laser wave network, satellite, mobile and/or other similar networks are implemented to transfer data (e.g., software, data acquired by sensors, instructions, firmware, an embedded component and/or type of functional component) between a server (e.g., Webserver, BLADE server, etc.) and one or more (e.g., 10 or more, 100 or more, 1000 or more, 10,000 or more, 100,000 or more, 1,000,000 or more) PCs and/or simulation devices. In some embodiments, a plurality of simulation devices are connected (e.g., via USB or wireless (e.g., WIFI, ZIGBEE, etc.) hub) to a single PC that has access to a server. Several preferred embodiments are described herein. However, it should be noted that communication systems, in the context of this invention, are interchangeable and may relate to various schemes of cable, fiber optics, microwave, radio, laser and similar communications or any combinations thereof.

In some embodiments, the present invention provides a server that manages the operational and functional parameters of one or a plurality of PCs and/or simulation devices connected thereto (e.g., in real-time). The present invention is not limited by the type of server utilized. Indeed, a variety of servers are contemplated to be useful in the present invention including, but not limited to, an application server, communication server, database server, file server, local PC, and standalone servers. In a preferred embodiment, the server is a Web server (e.g., a server that HTTP clients connect to in order to send data and/or commands and receive data and/or responses). In some embodiments, a server (e.g., a local PC serving multiple other local PC clients) connects to a separate server that manages the operational and functional parameters of one or a plurality of PCs and/or simulation devices connected thereto (e.g., in real-time). In some embodiments, a server is in communication with (e.g., is connected to) a Storage Area Network (SAN) (e.g., a high-speed subnetwork of shared storage devices (e.g., that comprise a disk, disks, tape or other computer readable form for storing data)). In some embodiments, SAN architecture allows one or more (e.g., 2, 5, 10, 15, 25, 35, 50, or more) storage devices to be available (e.g., accessible) to one or more servers (e.g., interconnected servers (e.g., on a LAN or WAN)). In some embodiments, because SAN allows data storage separate from a server, such a configuration is contemplated to allow increased efficiency of one or more servers (e.g., providing a quicker and more enjoyable experience for a plurality of users). A SAN also permits redundancy (e.g., of data (e.g., user performance data and/or certification) storage), thereby providing a more reliable system.

Accordingly, in some embodiments, the present invention provides a system as depicted in FIG. 1. In FIG. 1, a server 1 (e.g., connected to the internet) housing software 2 (e.g., a training program, a testing program, and/or data processing program (e.g. for determining user performance (e.g., identifying users that achieve or fail performance criteria for certification))) is accessible (e.g., via the internet) by one or a plurality of personal computers 3 (PC) that is connected to a simulation device 4 (e.g., a mannequin). In some embodiments, the PC 3 comprises software 5 (e.g., mannequin driver software and/or communication software (e.g., for sending and/or receiving information to and/or from the mannequin 4 and/or server 1)). In some embodiments, data 6 is acquired from the simulation device 4 by software 5 housed on the PC 3. In some embodiments, software 5 housed on the PC 3 permits the server 1 to download data 7 (e.g., testing data (e.g., a testing program), training data (e.g., a training program), performance data, certification data, etc.) from the server 1 to the PC 3 and/or simulation device 4. In some embodiments, software 5 housed on the PC 3 uploads raw data 8 (e.g., un-processed data) from the simulation device 4 onto the server 1. In some embodiments, software 5 on the PC 3 processes data 6 acquired from the simulation device 4 prior to uploading the processed data 7 onto the server 1. The present invention is not limited by the type of data 6 acquired from the simulation device 4. Indeed, a variety of types of data 6 can be acquired including, but not limited to, data obtained from one or more sensors 9 present on or within the simulation device 4 (e.g., head tilt sensors, pulse sensors, compression sensors, ventilation sensors, location sensors (e.g., for an AED device), charge sensors, etc.) user performance data, and sensor status data. Similarly, the present invention is not limited by the type of data 6 sent from the PC 3 to the simulation device 4. Indeed, a variety of types of data 6 can be sent from the PC 3 to the simulation device 4 including, but not limited to, real-time instructions from the server (e.g., related to simulation device activity). In some embodiments, a simulation device 4 comprises means 10 for communicating and/or connecting with a PC 3. The present invention is not limited by the means 10 for communicating and/or connecting. Indeed, a variety of different means 10 for communicating and/or connecting are contemplated including, but not limited to, via cable (e.g., USB type I, USB type 2, USB type 3, serial cable, etc.) and/or wireless (e.g., infrared, BLUETOOTH, WIFI (e.g., IEEE 802.11a, b, g, p, etc), WIMAX, ZIGBEE (e.g., ZIGBEE wireless networking tools), etc.) technologies.

In some embodiments, the present invention provides remote data accumulation, data processing and/or down linking instruction (e.g., user pass/fail instructions, certification/non-certification instructions, etc.) by the server (e.g., Web server). In some embodiments, the invention provides remote maintenance, upgrade, performance tracking, sensor diagnostic testing, tuning (e.g., of a sensor or other component of the simulation device), and/or adjustment of a simulation device connected to a PC and/or AED simulation device (e.g., from a remote location). In some embodiments, an upgrade, tuning, information and/or an adjustment may be made to an interactive simulation device (e.g., mannequin) via upgrading software on the remote PC and/or AED simulation device and/or on the simulation device connected thereto (e.g., in some embodiments, the remote PC transfers the upgrade, tuning, information and/or an adjustment to the simulation device (e.g., mannequin)) In some embodiments, an upgrade, tuning, information and/or an adjustment may be made to an interactive simulation device (e.g., mannequin) and/or AED simulation device via upgrading software on the AED simulation device and/or mannequin connected thereto via uploading the upgrade, information and/or adjustment to the AED simulation device via a USB drive (e.g., thumb drive). Thus, in some embodiments, real time information can be gathered by a server (e.g., Web server) from a PC and/or simulation device connected thereto, processed by the remote server (e.g., by software housed on the server), one or more instructions generated by the server, and the instructions delivered, all in real-time, to the PC and/or simulation device (e.g., mannequin) connected thereto (e.g., to alter the performance of the mannequin (e.g., to simulate a testing condition (e.g., to alter mannequin heart rate, breathing rate or other detectable parameter)) perceived by user of the system (e.g., to test user's reaction to a given situation)).

Thus, in some embodiments, the present invention provides a method of providing the same, a substantially similar, a similar, and/or a unique set of experimental datasets (e.g., questions, training exercises, testing exercises, simulated exercises, etc.) to a plurality of users of the system (e.g., in real-time). In some embodiments, the present invention provides a system capable of analyzing the performance of a plurality of users simultaneously (e.g., in real-time). In some embodiments, the present invention provides a system capable of analyzing the performance of a plurality of users over a period of time (e.g., one or more hours, a day, a week, a month, a year or more). In some embodiments, the present invention provides a method of enhancing user examination/performance by implementing simulations and/or testing scenarios that are configured to represent statistically relevant scenarios (e.g., as determined epidemiologically and/or via processing of user performance (e.g., using software run by a server of the present invention)) that are down linked from the server to the PC and/or simulation device connected thereto.

In some embodiments, the present invention provides an adaptable/flexible communications scheme that permits continuous real-time communications between a remote server (e.g., Web server) and a plurality of PCs and/or simulation devices (e.g., present at one or a plurality of locations). Thus, in some embodiments, testing scenarios can be provided (e.g., by software housed within the server and/or on a local PC) that simulate (e.g., using a plurality of PCs and/or simulation devices) an event involving a plurality of individuals requiring immediate life support care (e.g., due to a natural disaster, terrorist event, accident, chemical exposure, etc.). In some embodiments, the needs of each simulated individual (e.g., as represented by the simulation devices (e.g., mannequins)) requiring care is coordinated with the other members of the plurality of simulated individuals (e.g., providing a more life-like scenario for first-responders). In some embodiments, the simulation device (e.g., mannequin) is configured (e.g., with sensors, software, firmware, etc.) to share information (e.g., user performance) with a PC that is monitored, analyzed and acted upon (e.g., in real-time) by software housed on a server (e.g., a Web server).

In some embodiments, the present invention provides a method of using an accessible server (e.g., Web server) to collect, manage and process information (e.g., user performance data located on a USB drive (e.g., of an AED simulation device described herein). In some embodiments, an accessible server (e.g., Web server) is used to manage, troubleshoot, update and/or otherwise alter operational and functional parameters of a PC, AED simulation device, (e.g., via downloading information to a USB drive and inserting the drive into the AED simulation device) and/or simulation system connected thereto (e.g., in real-time). For example, the present invention provides a method of remote diagnosis, maintenance, upgrade, performance tracking, tuning and/or adjustment of a PC and/or a simulation device connected thereto as well as the component parts (e.g., software, sensors, firmware, etc.) of each. Thus, in some embodiments, the present invention provides a method of providing a training, testing and/or evaluation session to a plurality of users in the same or different remote location (e.g., at the same time) wherein the training, testing and/or evaluation session is the same or different between the plurality of users. The flexibility of this type of process allows the ability to curb the propensity for cheating (e.g., on a written portion of a test) by providing altered formatting (e.g., order of questions presented) for multiple users. The process also allows the for the administration of a plurality of training, testing and evaluation sessions limited only by the instructions found within software housed on an accessible server, or alternatively, housed in a limited access (e.g., administrator only access) portion of a PC. The systems and methods of the present invention also allow a server to compile a database of user performance (e.g., receiving and storing user information and performance data (e.g., for statistical analysis (e.g., that can be presented to a body or organization (e.g., American Heart Association, American Red Cross, and/or National Safety Council) responsible for the administration of certification examination for one or more procedures for which the systems and methods of the present invention are utilized (e.g., CPR, AED, etc.)))) from one or a plurality of PCs, AED simulation devices (e.g., from USB drives compiling data (e.g., user performance data) therefrom) and/or simulation systems connected thereto. The server may comprise software applications for monitoring, analysis and/or characterization of data accumulated by an AED simulation device (e.g., user performance data stored on a USB drive). Examples of software applications for monitoring, analysis and/or characterization of data include, but are not limited to, identification of trends of key diagnostic parameters and identification of CPR and/or AED usage parameters that need to be adjusted.

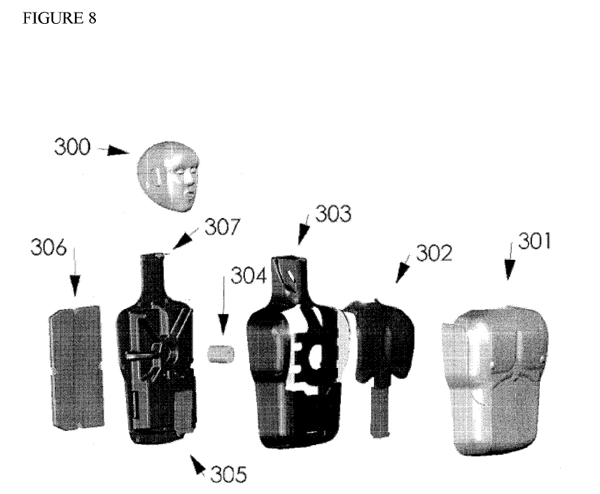

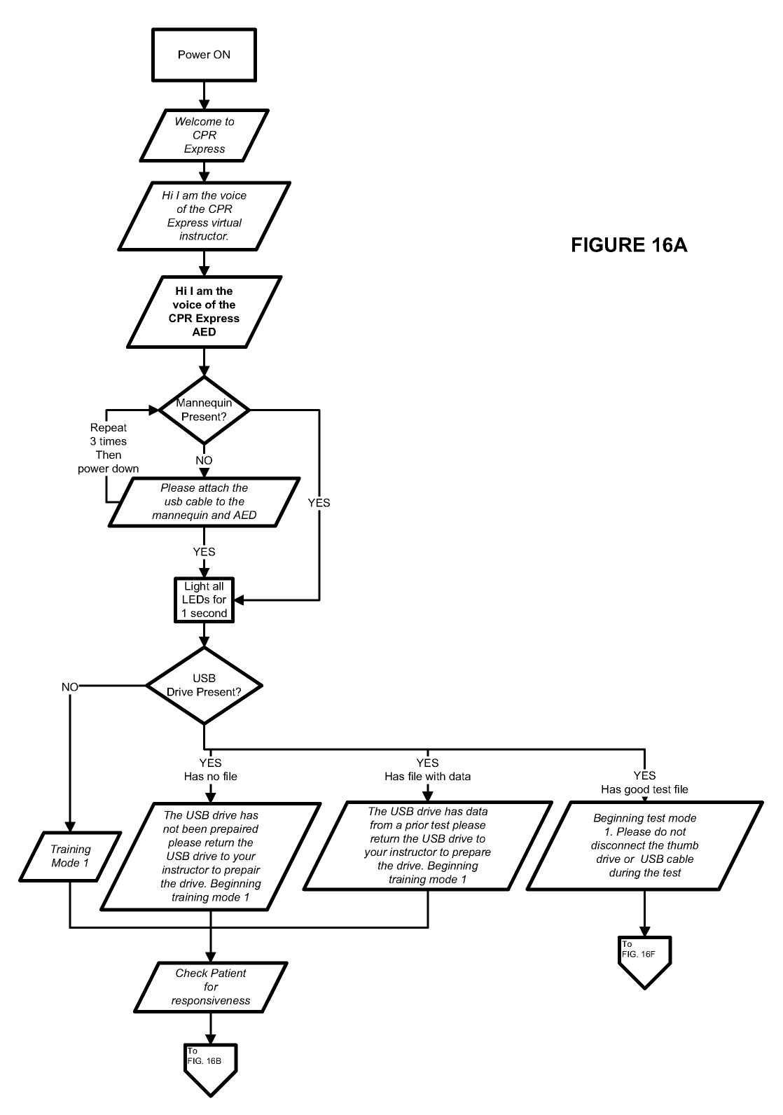

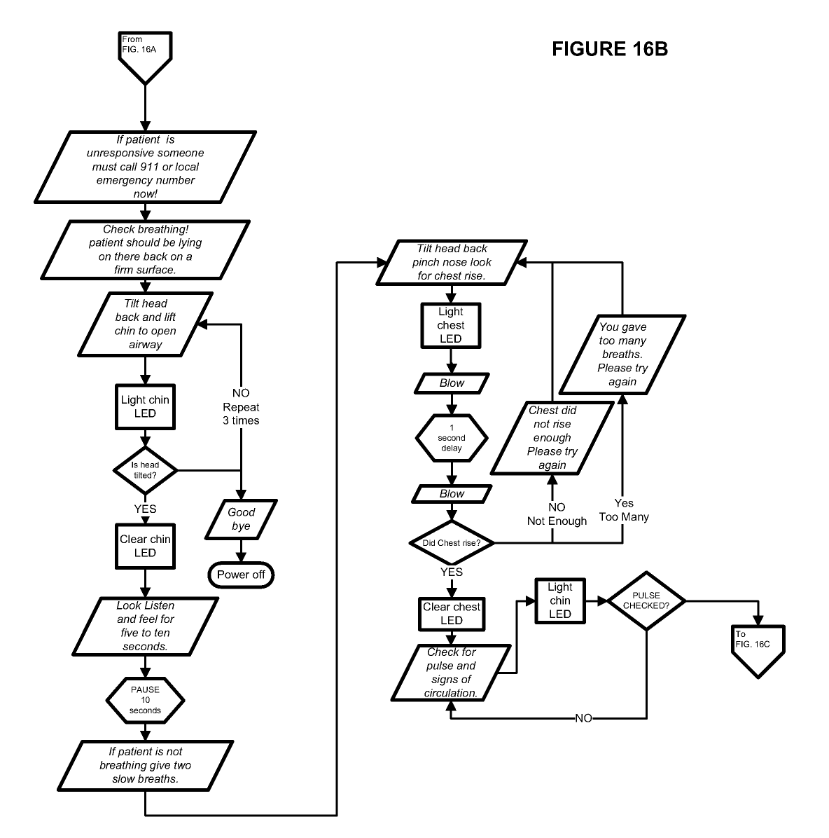

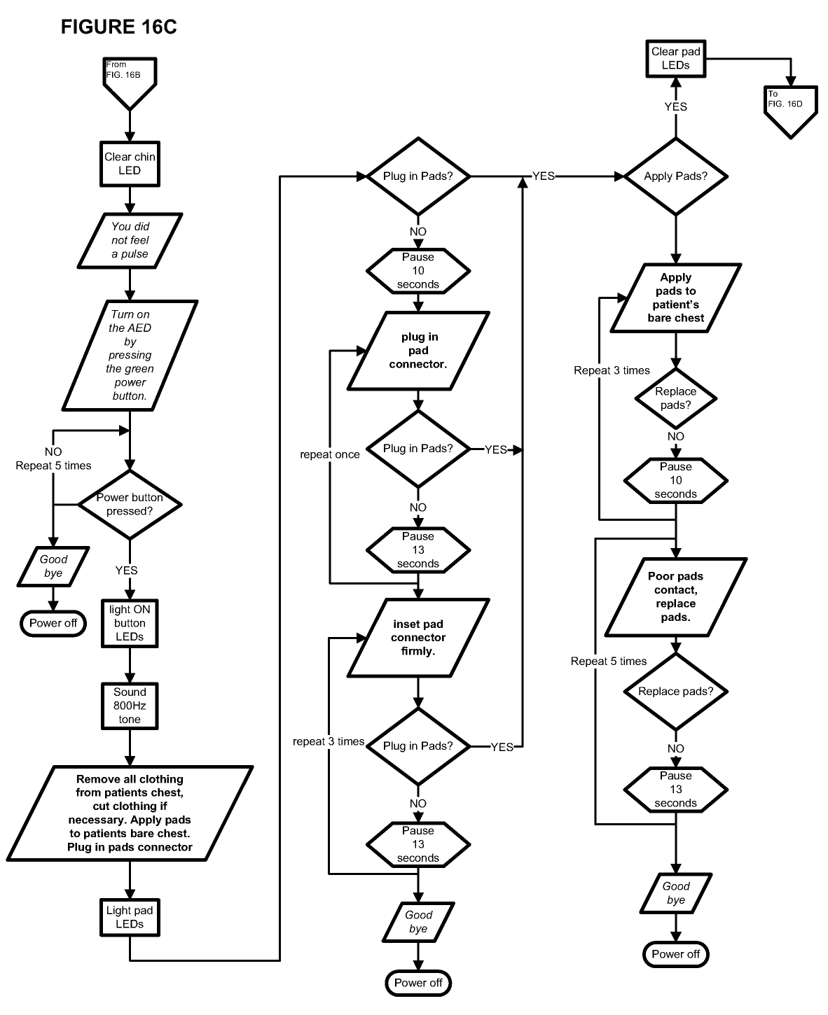

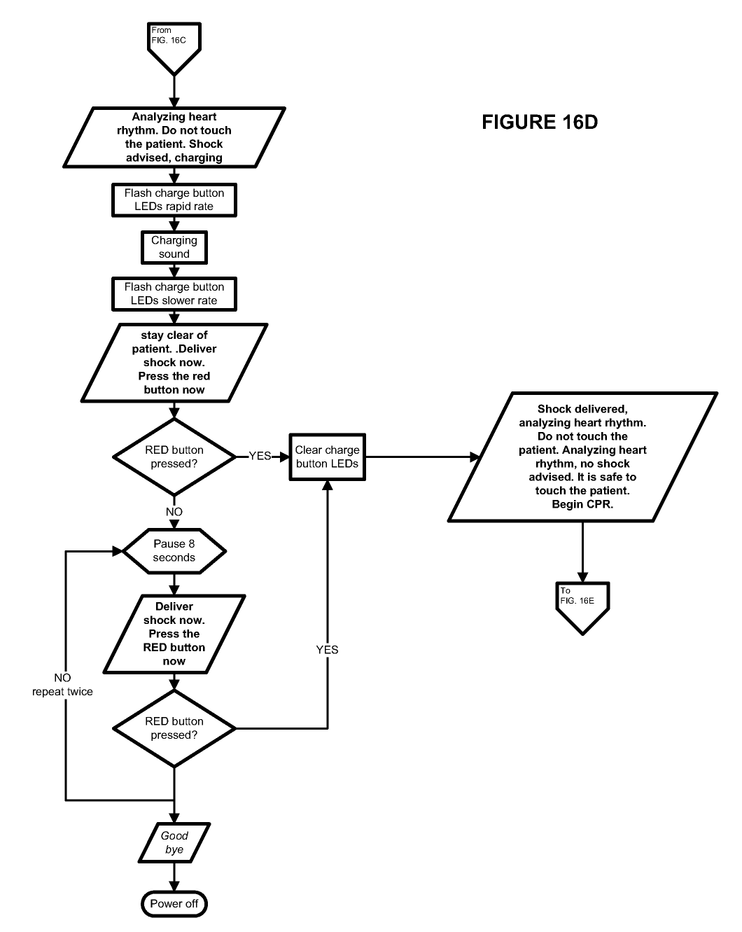

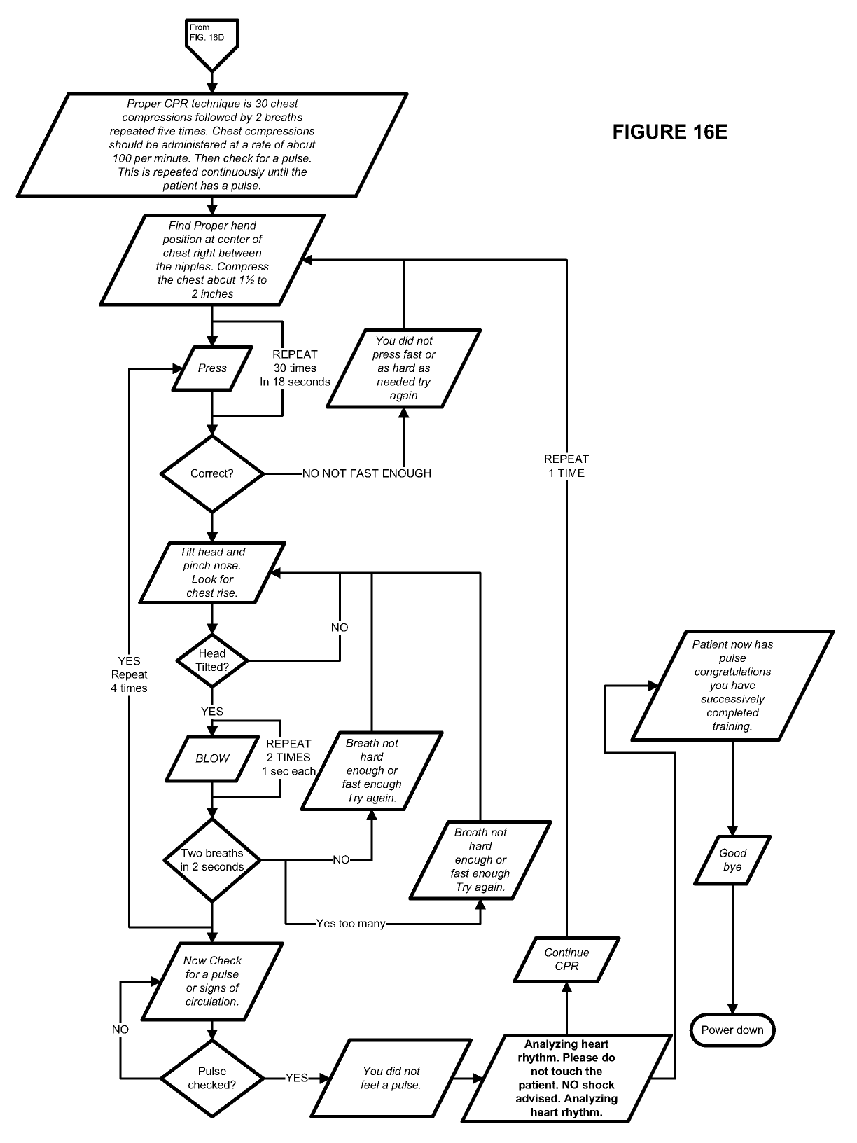

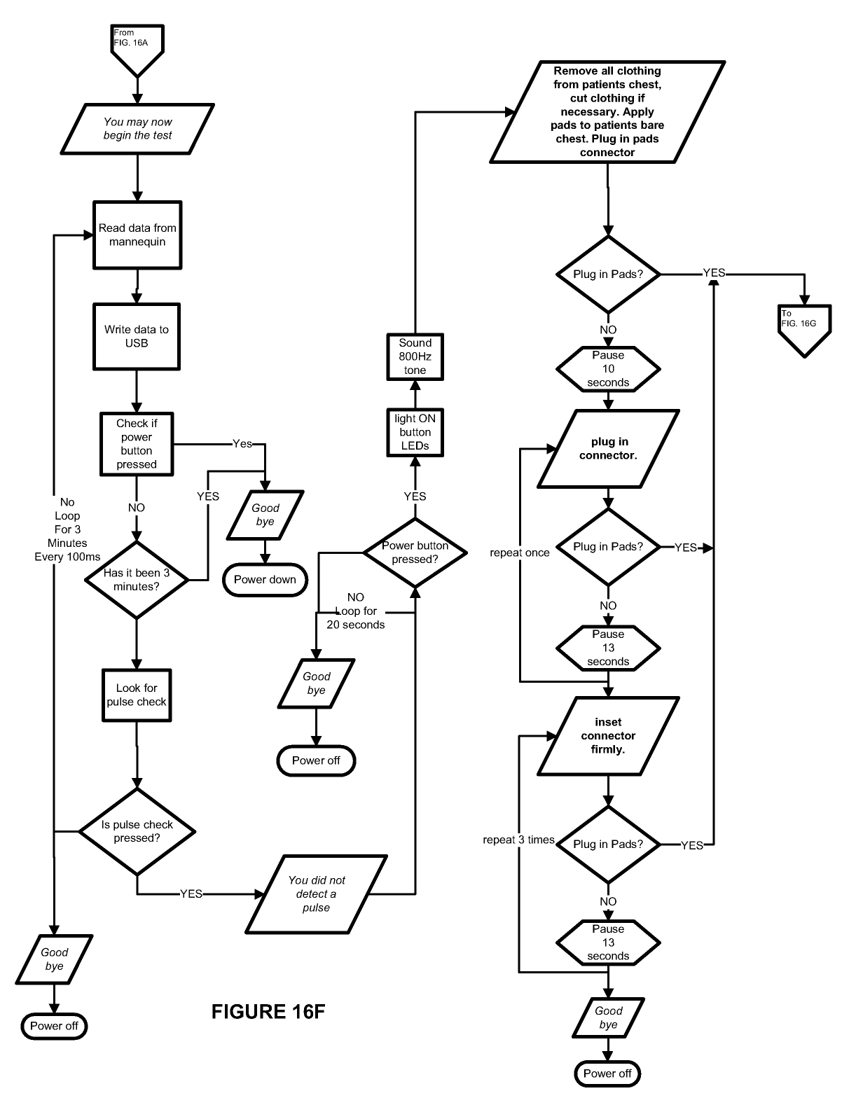

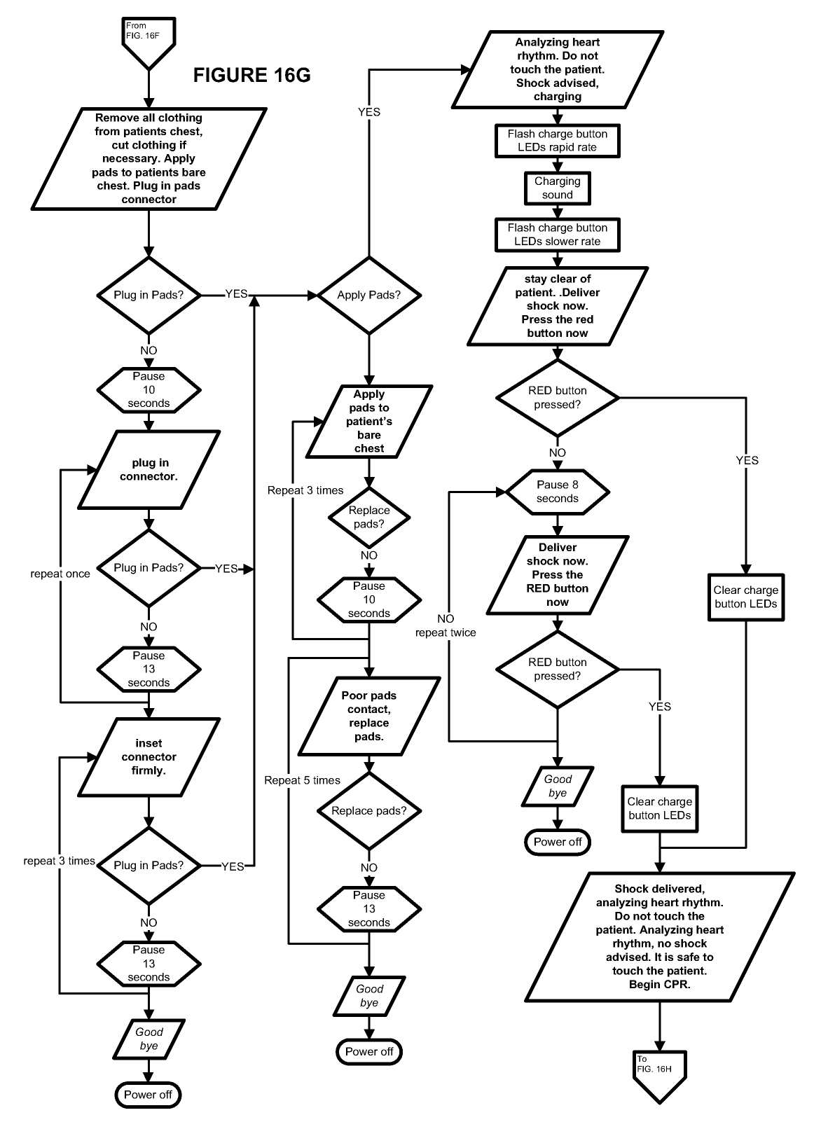

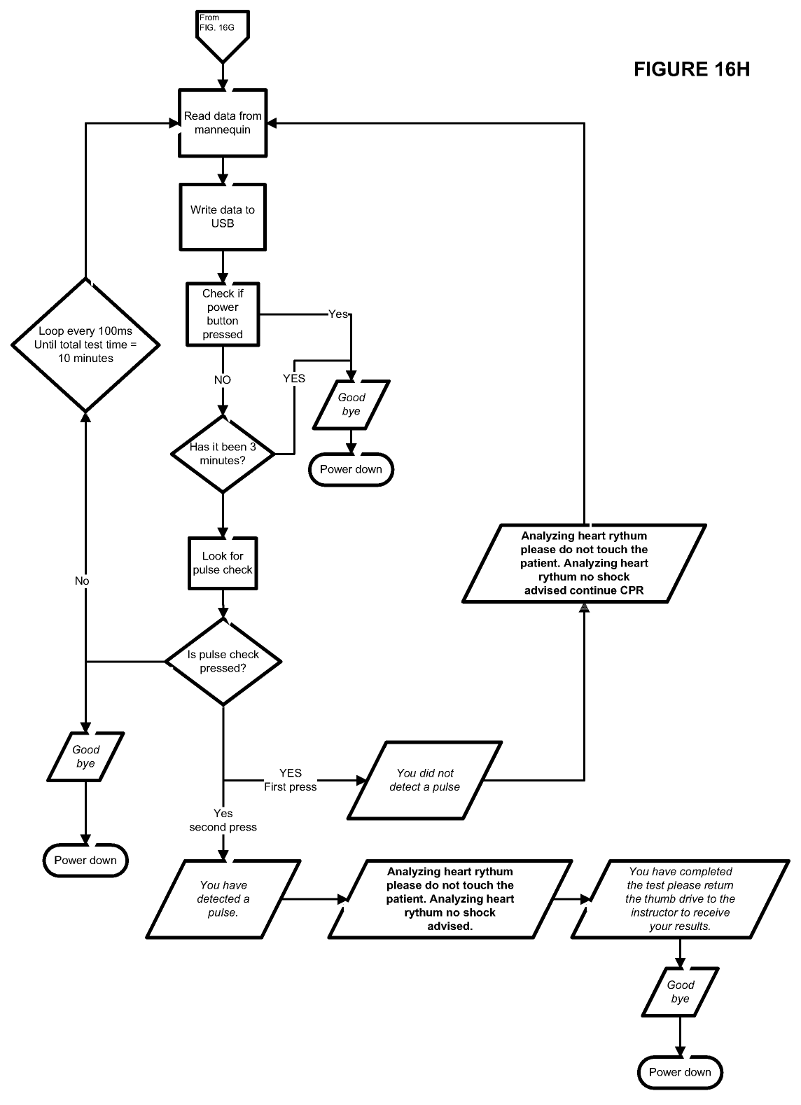

In some embodiments, a user of the systems and methods of the present invention can print out (e.g., upon successful completion of a training, testing and/or evaluation session) a certificate (e.g., authorized certificate, official certificate, certificate recognized by a body and/or organization (e.g., American Heart Association, American Red Cross, and/or National Safety Council)) responsible for administering training, testing and/or evaluation/certification) upon successful completion of a training, testing and/or evaluation/certification session. Examples of embodiments utilizing a system and methods of the invention are provided in FIGS. 8 and 16. For example, a server comprises software that analyzes and/or evaluates user performance and determines whether a trainee and/or test taker has achieved satisfactory performance to pass a predefined performance criteria. The server also comprises software to generate user certification (e.g., in the performance of CPR and/or AED usage). In some embodiments, the server comprises software that distributes user performance date and/or certification information to one or more certification authorities (e.g., the American Heart Association, American Red Cross, and/or National Safety Council, and/or equivalent national organizations in non-US territories, e.g., Europe, Asia, South America, etc.).

For example, upon completion of a simulated training module corresponding to material presented in a training module (e.g., See FIG. 16), the trainee and/or test taker is required to complete a test. If the trainee and/or test taker answers a certain percentage correctly (e.g., at least eighty percent of the questions correctly), upon loading the user performance data located on a USB drive onto a server, software present on the server evaluates the test results and generates certification data. The server distributes the trainee and/or test taker's certification data to a certification authority. Similarly, certification data can be made accessible to an administrator and/or test distributor.

In some embodiments, the present invention provides systems and methods of altering (e.g., upgrading) a simulation device and/or sensors and/or components thereof (e.g., software, firmware, activity, etc.). Thus, in some embodiments, the present invention provides an interactive system for training, testing and/or evaluating a user to carry out a particular task (e.g., CPR, administration of AED devices, etc.). For example, a simulation device and/or sensors and/or components thereof may be configured to communicate in real-time with a server and/or software housed therein (e.g., directly or via a PC (e.g., software run on the PC)).

Thus, the present invention provides the advantage of remote administration, maintenance, troubleshooting and upgrading. In some embodiments, a system of the present invention provides a scheme of communication that permits remote maintenance, debugging and/or analysis of a PC and/or simulation device connected thereto and/or component parts thereof (e.g., software, firmware, sensor, etc.). For example, in the event a component or software defect is noted, the system is able to ascertain whether a fix (e.g., a remote fix) is possible. In the event a defect is detected and a remote fix does not exist, the system may generate an alert that is broadcast on the PC and/or simulation device connected thereto thereby notifying a user on a real-time basis. In the event a defect is detected and a fix (e.g., a remote fix) does exist, the system may down link (e.g., download) information (e.g., source code, software, firmware, etc.) from the server to the PC and/or simulation device connected thereto. In some embodiments, while executing this process, the server may review and/or analyze (e.g., in real time) one or more types of data (e.g., including, but not limited to, usage logs, error logs, power and battery status, data base integrity and/or the mean time between failures status of all the significant and relevant components, user performance data and integrity of same, and software status (e.g., that is stored within a database present on the server and/or that is a component of the PC and/or simulation device connected thereto that is continually monitored in real-time by the server)).

In some embodiments, systems and methods of the present invention are configured to provide significant compatibility and scalability to other web-based applications (e.g., telemedicine and emerging web-based technologies (e.g., tele-immersion)). For example, a system of the present invention may be adapted to webtop applications in which a webtop unit may be used to uplink one or more simulation devices to a server (e.g., Web server) for information exchange between the simulation devices and the remote server. In some embodiments, the information collected may be used to identify the need and/or desire to refine and/or upgrade the system using the advanced web technologies.

In some embodiments, the systems and methods of the present invention provide the remote training, testing and evaluation of users (e.g., medical technicians, nurses, doctors, emergency medical persons, teachers, and persons desiring training) in the use, process and/or operation of a skill set (e.g., that can be simulated on a simulation device of the present invention (e.g., CPR, administration of an AED device, and/or other skill described herein)). In some embodiments, the user is trained using software-based simulated training exercises run on a server and emulated through the PC, or alternatively, run locally on a PC, AED simulation device and/or other simulation device connected thereto (e.g., a mannequin). As described herein, a system of the present invention permits an interactive training scheme (e.g., the user can proceed through a plurality of different training programs based on levels of difficulty and/or complexity housed on a server or locally on a PC or AED simulation device or USB drive connected thereto (e.g., permitting user to determine user's level of expertise and appropriate level at which to begin training)). Once the user feels that the user is adequately trained, user can choose to run one or more separate programs (e.g., that test and/or evaluate the user's performance in one or a plurality of different skills/procedures, e.g., as shown in FIG. 16)).

The present invention is not limited by the type of user performance data collected, analyzed, and acted upon (e.g., by software run by a server (e.g., that generates instructions for the PC, AED device and/or other simulation device connected thereto based upon analyzed data)). Indeed, a variety of user performance data can be collected, analyzed and/or acted upon including, but not limited to, the types of skills referenced in FIG. 16. Additionally, other components may be added for additional testing and/or monitoring scenarios including, but not limited to, other types of medical procedures that may be performed on a subject administered CPR and/or AED defibrillation (e.g., administration of an IV, assessment of injury, temperature reading, patient stabilization (e.g., physical and/or emotional), patient immobilization, etc.). The present invention is not limited by the type of processing of data accumulated by a PC and/or server, nor by the data (e.g., instructions) generated by such processing.

The present invention is not limited by the type of user of the systems and methods of the present invention. Indeed a variety of different types of users may use, market, require and/or promote training, testing and/or certification by systems and methods described herein. For example, in some embodiment, a user may be an organization that requires its members to use systems and methods described herein (e.g., in order to become certified in a specific procedure (e.g., CPR and/or use of an AED). Examples of organizations that may use the systems and methods of the present invention include, but are not limited to, hospitals, schools, fire departments, police departments, churches, the coast guard, a branch of the military (e.g., Army, Navy, Air Force, Marines), towns, cities, states, day care centers, retirement homes and/or communities, amusement and/or theme parks, airlines, cruiselines, etc. Similarly, a number of different types of individuals (e.g., within an organization, or, not affiliated with an organization) may utilize systems and methods of the present invention (e.g., for certification in a specific procedure (e.g., CPR and/or AED usage) including, but not limited to, teachers, emergency medical technicians, police officers, fire department employees, nannies, hospital employees (e.g., nurses, doctors, staff, etc.), parents, day care providers, lifeguards, parents, etc. In like manner, it is contemplated that bodies and/or organizations responsible for accreditation and/or certification for certain procedures (e.g., CPR, AED usage, etc.) may require and/or recommend the use of systems and methods of the present invention. Examples of such bodies and/or organizations include, but are not limited to, American Heart Association, American Red Cross, and National Safety Council.

The present invention is not limited by the type of simulation device utilized in the systems and methods of the present invention. Indeed, a variety of simulation devices are contemplated including, but not limited to, those described in U.S. Pat. Nos. 5,590,057; 5,593,306; 5,853,292; 6,157,808; 6,503,087; 6,527,558; and 6,193,519, each of which is hereby incorporated by reference in its entirety for all purposes. In some embodiments, a simulation device may be “rented” by a user from a centralized rental facility, allowing a user to use the systems and methods of the present invention at any location (e.g., at home) and at any time desired by the user.

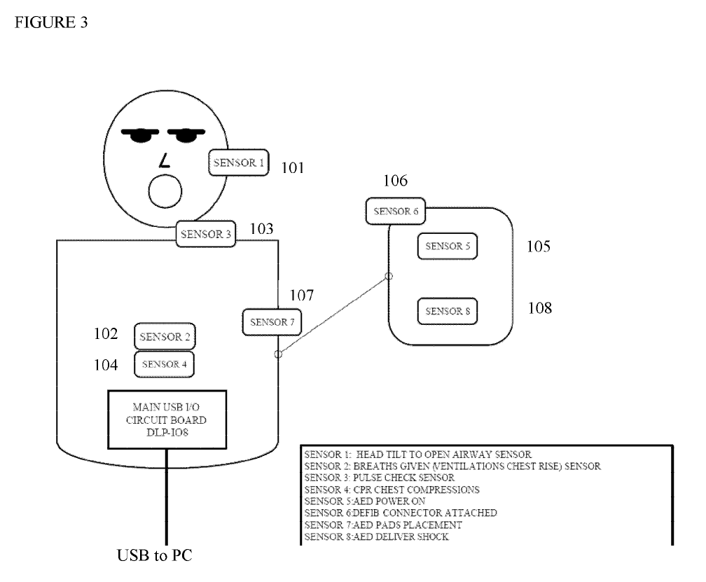

In some embodiments, the simulation device is in accordance with a device shown in FIGS. 3, and 10-15. For example, in some embodiments, a simulation device comprises a head tilt sensor 101 (e.g., comprising a magnetic prox sensor (e.g., hall effect sensor (e.g., in the mannequin head (e.g., to record head tilt and/or patient airway))), a rise (e.g., ventilation chest rise (e.g., hall effect)) sensor 102, a pulse (e.g., touch pressure (e.g., tact switch)) sensor 103, a compression (e.g., CPR chest compression (e.g., hall effect)) sensor 104, a momentary (e.g., power on/off, deliver shock, etc. (e.g., hall effect)) sensor 105, 108, a nose pinch (e.g., tact switch) sensor 110, and/or a location/contact (e.g., magnetic prox (e.g., for an AED device (e.g., hall effect))) sensor 106, 107.

The simulation device (e.g., mannequin and/or AED simulation device) may comprise a guide system that assists a user in training with a system of the present invention. The present invention is not limited by the type of guide system. Indeed, a variety of guide systems may be utilized including, but not limited to, a voice prompt guide system, a train to light guide system, a brail guide system, any combination of one or more of the above, and/or other type of guide system. Thus, the present invention provides a system that assists users (e.g., trainees or other types of users that have not been able to train and/or learn skills on conventional systems) in training with the system and learning CPR and/or AED usage. For example, the guide system of the present invention provides effective means of training individuals with visual impairment, hearing impairment, learning disabilities, or other type of disorder that would impair such a user from training and/or learning skills associated with CPR and/or AED usage using conventional devices and/or systems.

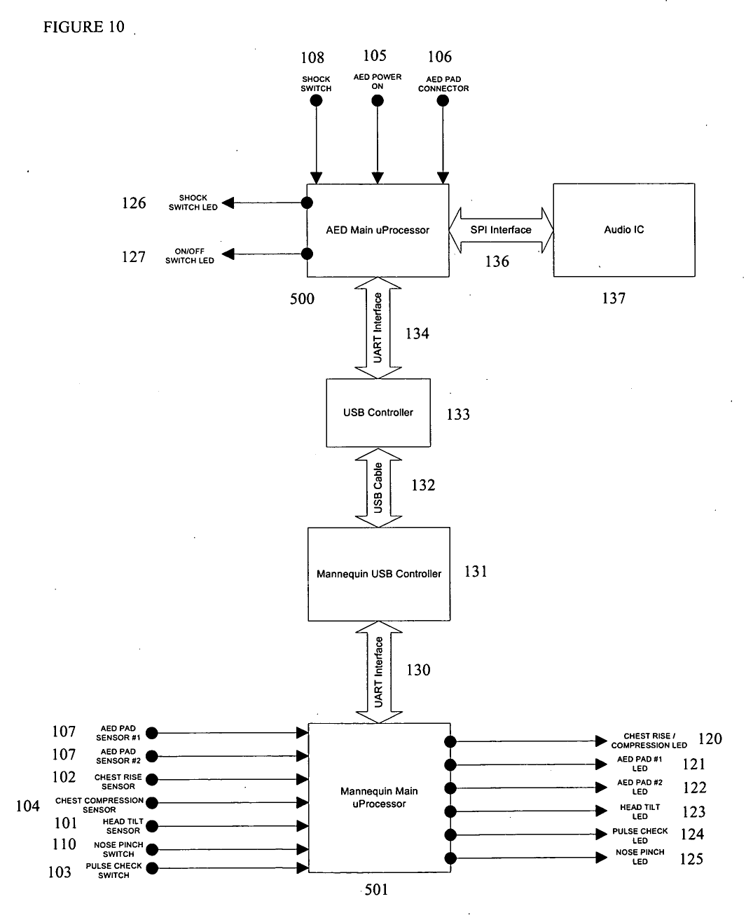

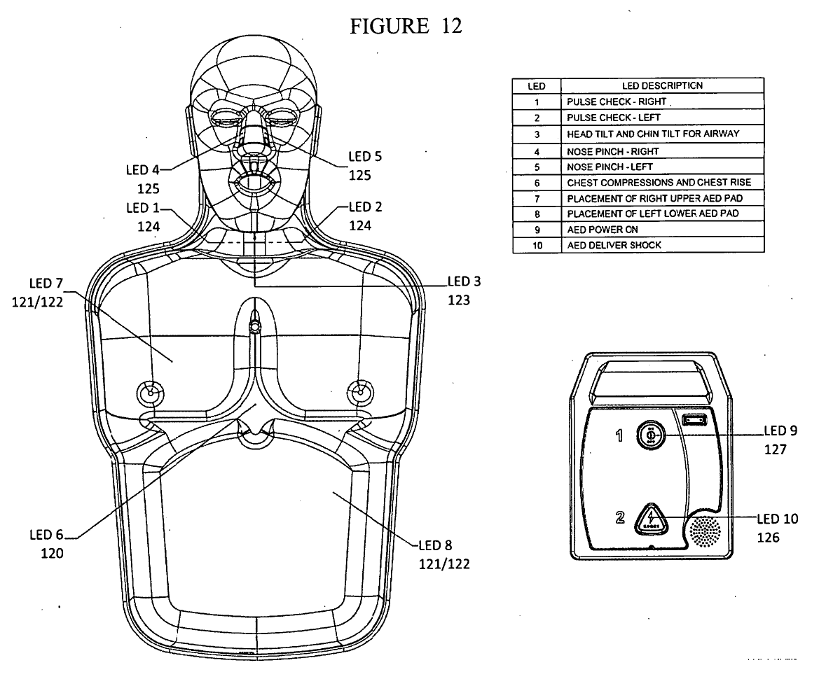

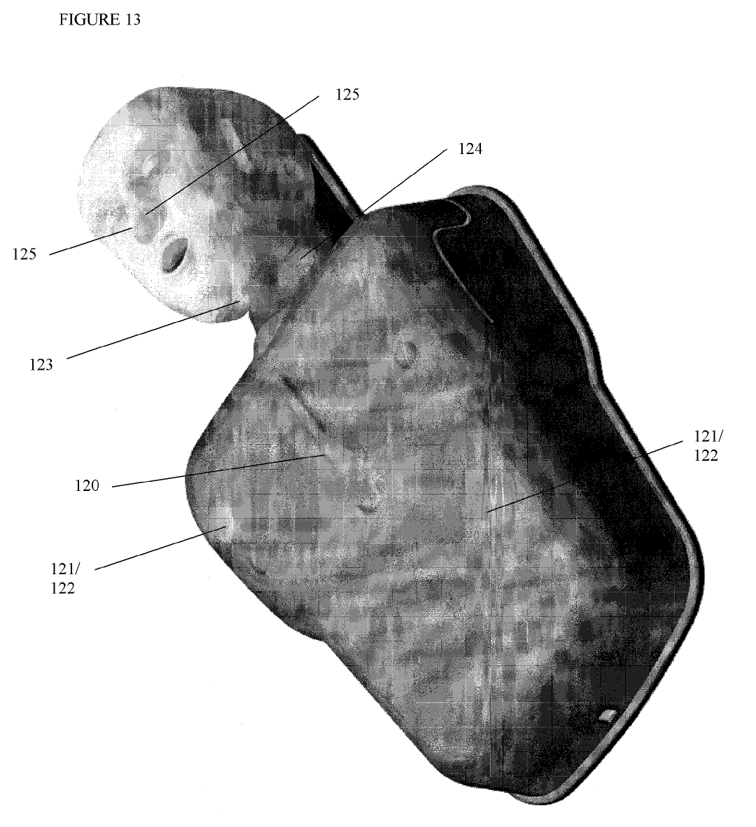

For example, the simulation device (e.g., mannequin and/or AED simulation device) may, as shown in FIGS. 10, 12, and 13, comprise one or more LEDs 120-127 The LEDs 120-127 are utilized to assist a user in training with a system of the present invention. The present invention is not limited by the number of LEDs nor the placement of the LEDs (e.g., that are illuminated by a system of the invention in order to train and/or guide a user of the system). FIGS. 12 and 13 describe various LEDs 120-127 and the locations thereof within a mannequin and/or AED simulation device in some embodiments of the invention. In some embodiments, an LED is placed at one or more (e.g., each) data capture test locations (e.g., the location of a sensor (e.g., as described in FIGS. 3, 11 and 12).

In some embodiments, the present invention provides a system (e.g., CPR and/or AED training and/or testing system) comprising a train to light guide system. In some embodiments, the train to light guide system comprises a protocol that places a light indicator at a location where a user (e.g., a trainee learning CPR and/or AED usage with a system of the invention) needs to perform an action (e.g., of CPR and/or AED usage). The light is illuminated when a user needs to perform the action and extinguished when the proper action has been completed.

In some embodiments, the present invention provides a brail guide system (e.g., CPR and/or AED training and/or testing system) comprising brail prompts (e.g., for a visually impaired trainee and/or test taker). Thus, the present invention guides visually impaired (e.g., legally blind) users to known locations for training and/or testing (e.g., the location of an audible instruction (e.g., voice prompt) indicating an action that needs to occur (e.g., for CPR and/or AED usage)). Thus, blind users can familiarize themselves with locations prior to and/or during CPR and/or AED usage. For example, the present invention provides that components of the mannequin and/or AED device (e.g., the on/off button) have brail describing the component (e.g., the on/off button is identified in brail as an on/off button, or the pulse check button is identified in brail as the location to check the pulse).

In some embodiments, a mannequin of the present invention comprises ribs (e.g., material utilized to construct the chest comprises cut-outs that simulate a humans ribs). In some embodiments, the size and/or placement of the ribs in the chest can be adjusted for compression force. Thus, in some embodiments, a mannequin device of the invention provides a more life-like and/or accurate simulation (e.g., of an actual human's (e.g., adult, child, elderly person, etc.) chest (e.g., when a user of the invention compresses the chest)).

In some embodiments, a translucent skin is placed over the chest. The present invention is not limited to any particular translucent material. Indeed, a variety of materials can be utilized including, but not limited to, poly vinyl chloride (PVC) plastomer (e.g., produced by molding (e.g., dip molding)) or other similar materials (e.g., composites (e.g., that are durable and/or playable). The translucent nature of the skin allows light from the LEDs to transmit through it. In some embodiments, the skin is attached to the mannequin body by stretching it over the outer ribs. The skin may be held in place by a channel in the outer perimeter of the mannequin body.

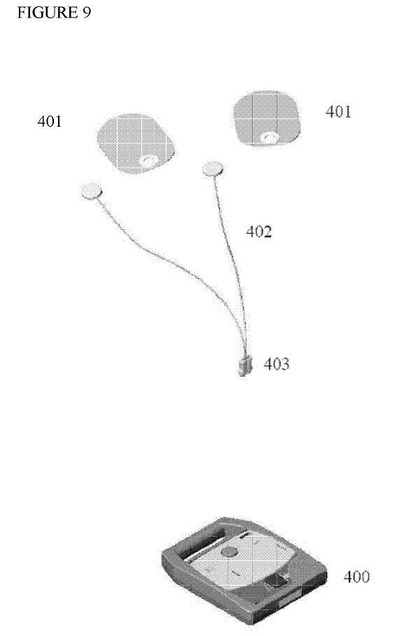

In some embodiments, the present invention provides an AED simulation device (e.g., training unit) assembly 400 (e.g., for use with a mannequin described herein). Preferably, an AED simulation device 400 (e.g., training and/or testing unit) is a life-like model of a real AED (e.g., currently available or yet to be generated). An example of an AED unit of the present invention is shown in FIGS. 9, 11-12, and 14-15. In some embodiments, the AED unit 400 is used together with a shock pad assembly 420 comprising a pair of defibrillator pads 401, a pair a wires 402, and/or an attachment 403. In some embodiments, the AED training unit assembly 400 comprises power on and off buttons. In some embodiments, the AED unit comprises sensors described herein (e.g., to detect pad placement and/or movement of on and/or off buttons). In some embodiments, an AED training unit comprises a speaker (e.g., to simulate a real AED's instructional voice prompts and/or to provide training instructions). An AED unit may also comprise one or more sensors. Exemplary sensors include, but are not limited to, a hall effect sensor, a switch sensor, a momentary tactile button sensor (e.g., to record the powering on/off of the unit); a molex-type connector sensor (e.g., that records the plugging in of the AED defibrillation pads connector); and/or a momentary tactile button sensor (e.g., that records the deliverance of an AED shock if so instructed/advised).

In some embodiments, the present invention provides a system (e.g., for training, testing and/or certifying a subject (e.g., in the performance of AED usage and/or CPR)) comprising an AED device 400 comprising a microcontroller 500 (e.g., comprising a processor) in communication (e.g., bi-directional communication) with a mannequin comprising a microcontroller 501 (e.g., comprising a processor, as shown in FIGS. 10-15). For example, the present invention provides communication means 130-134 between a handheld AED simulation device (e.g., comprising a processor) and one or more simulation devices (e.g., a mannequin (e.g., comprising a processor and/or one or more sensors) connected thereto and/or components (e.g., controls and/or sensors) associated therewith. As shown in FIGS. 10-15, in one embodiment, a system comprises an AED device 400 (e.g., an AED simulation device (e.g., that simulates an actual AED (e.g., that simulates automatically diagnosing potentially life threatening cardiac arrhythmias of ventricular fibrillation and/or ventricular tachycardia in a subject and that treats/defibrillates the subject via the application of electrical therapy that stops the arrhythmia), but that does not actually provide electric therapy)). In some embodiments, the AED device 400 comprises a microcontroller 500 and firmware (e.g., that allows the AED device 400 to communicate with external components (e.g., a mannequin (e.g., components of the mannequin (e.g., LEDs, sensors, and/or other components of the mannequin etc.)). The AED device 400 may house one or more batteries for powering the system (e.g., in some embodiments, batteries of the AED simulation device 400 power both the AED device 400 and a mannequin (e.g., components of the mannequin (e.g., LEDs, sensors, and/or other components of the mannequin) run only off of the power of the AED's batteries). The AED device 400 may further comprise one or more USB ports/controllers (e.g., male and/or female ports). The USB ports may be utilized for communication with a USB (e.g., thumb) drive 321 (e.g., comprising information related to testing and/or training) or connection to a USB cable 132 that provides a communication means between the AED device microcontroller 500 and a mannequin microcontroller 501. The communication means between the AED device microcontroller 500 and a mannequin microcontroller 501 are not limited to a USB connection. Indeed, a variety of different means for communicating and/or connecting are contemplated including, but not limited to, via cable (e.g., USB type I, USB type 2, USB type 3, serial cable, etc.) and/or wireless (e.g., infrared, BLUETOOTH, WIFI (e.g., IEEE 802.11a, b, g, p, etc), WIMAX, ZIGBEE (e.g., ZIGBEE wireless networking tools), etc.) technologies.

The AED device 400 and mannequin may comprise one or more LEDs 120-127. As described herein, the LEDs 120-127 are utilized to assist a user in training with a system of the present invention (e.g., the LEDs provide a light signal to a user that is training with the system). The AED device 400 may further comprise one or more sensors (e.g., hall effect sensors, switch sensors, or other type of sensor) utilized to determine if a user (e.g., trainee, test taker, etc.) has properly performed one or more tasks associated with testing, training and/or certification using the system. For example, the AED device 400 may comprise a sensor associated with an AED power on button 105 (e.g., that detects whether a user has properly turned the AED device 400 on/off). The AED device 400 may comprise a sensor 106 associated with a port for connecting a shock pad assembly 420 comprising a pair of defibrillator pads 401, a pair a wires 402, and/or an attachment 403 (e.g., that detects whether a user has properly connected the shock pad assembly attachment 403 to the AED device 400). The AED device may also comprise a sensor associated with a shock button 108 (e.g., that detects whether a user has properly pushed the shock button). The AED device 400 may also comprise a speaker and an integrated circuit for audio 137 (e.g., to provide audio/voice instructions encoded in firmware housed on the AED device 400).

Firmware housed within the AED device 400 is configured to utilize the AED 400 and mannequin of a system of the present invention for training, testing and/or certifying a subject (e.g., in the performance of CPR and or AED usage). For example, as shown in FIG. 16, when a training mode is entered, voice prompts and LED illumination are utilized to indicate to a trainee the location where an action should take place. FIG. 16 provides examples of various tasks (e.g., a scenario) that a user can be trained to perform and/or tested for performance, including, but not limited to, attaching cables, checking pulse, tilting head, chest compression (e.g., rate of compression), nose pinching, defibrillator pad placement, general handling and use of the system, delivering a shock, etc. In some embodiments, a device of the invention comprises a multiple person voice training methodology (e.g., a two-person voice training system). For example, a device may comprise a plurality of voices (e.g., male, female, or other type of distinctive voices (e.g., in any language) made audible over a speaker of an AED simulation device, wherein one voice is a virtual instructor (e.g., a female voice that assists a student/trainee in learning how to perform CPR and/or AED usage), and wherein a second voice is a voice of the AED simulation device (e.g., a male voice guiding a trainee and/or user through defibrillation and usage of the device). The usage of multiple voices is indicated in FIG. 16 (e.g., a voice of a virtual instructor for CPR is shown in italics and a voice of an AED simulation device is shown in bold).

Thus, in some embodiments, the present invention provides a system configured to relay information (e.g., instructions and/or performance data) between one or more processors (e.g., housed on a personal computer, AED device 400, or other device) and one or more additional processors (e.g., housed on a simulation device (e.g., a mannequin comprising one or more sensors (e.g., hall effect sensors and/or switch sensors associated therewith). In some embodiments, user performance data (e.g., training data and/or testing data) is stored on a USB drive (e.g., thumb drive) 321 connected to the AED device 400. In some embodiments, a user (e.g., trainee and/or test taker) of a system of the present invention receives a thumb drive 321 (e.g., from an administrator) for use with the system. The USB drive 321 can be configured (e.g., by an administrator/instructor) prior to use by a trainee and/or test taker. For example, an administrator/instructor can connect the thumb drive to a computer with internet connection and log onto a website configured to provide test and/or training information to the thumb drive 321. Once connected to the website, the administrator/instructor may be required to register with a user name and/or password. Once logged in, the website prepares the thumb drive 321 after the administrator/instructor requests the website to prepare the thumb drive 321 (e.g., by pressing the appropriate button/link on the website). Once requested, the website software looks for a drive with a volume of a particular label (e.g., “CPREXPRESS”). When found, the Website can be configured to assume that the drive with the particular label (e.g., “CPREXPRESS”) is the thumb drive 321. The Website can be configured to identify if there is a test file on the drive (e.g., with the name of “CPRTEST.CSV”). If there is no test file, then the thumb drive can be erased and a test file (e.g., with the name “CPRTEST.CSV”) can be loaded onto the thumb drive 321. If there is a test file already on the thumb drive 321 (e.g., with the label of CPRTEST.CSV) then the file is opened for reading. In some embodiments, the test file (e.g., labeled CPRTEST.CSV) contains test information including, but not limited to, test identification number, date, time, etc.). In some embodiments, one or more types of written tests are administered together with (e.g., before, during and/or after) training and/or testing with a device of the present invention. In some embodiments, correlating test identification number (e.g., on a written test and/or training and/or testing using a device of the present invention (e.g., utilizing a USB drive and corresponding drive filename to provide and/or store data (e.g., user performance data)) is utilized to prevent cheating.

In some embodiments, when the thumb drive 321 is placed into the AED 400 and the AED 400 is powered on the microcontroller 500 in the AED looks for a test file (e.g., with the name of “CPRTEST.CSV”). If the microcontroller 500 in the AED 400 does not find the file a voice prompt from the AED 400 notifies the user that the thumb drive 321 has not been prepared and that the user should take it to the administrator/instructor for preparation. If the AED microcontroller 500 finds a test file (e.g., named “CPREXPRESS.CSV”) it opens the file and reads it. If the test file does not end in “MCrLf” then the AED microcontroller 500 assumes there is test data in the file from a previous test. A voice prompt then notifies the user that the USB drive 321 contains data from a prior test and that the user should return the drive 321 to the administrator/instructor to have the drive 321 prepared (e.g., at this stage, the AED would begin training mode (e.g., as shown in FIG. 16)). If the AED microcontroller 500 finds a test file on the thumb drive 321 that contains a file called “CPREXPRESS.CSV and that ends with “MCrLf” then the AED microcontroller 500 notifies the user that it is beginning a test mode and that the user can begin the test. The microcontroller 500 records test performance data to the test file (e.g., labeled “CPRTEST.CSV”) on the thumb drive. Various pieces of information that can be stored on in test file include, but are not limited to, test scenario (e.g., pulse or no pulse, shockable EKG heart rhythm verses non-shockable heart rhythm, multiple ordering schemes for these and other conditions etc), AED and/or CPR test portions, sensor positions (e.g., 1=closed; 0=open), head tilt sensor position, pulse check sensor position, chest rise sensor position, chest compression sensor position, AED pad(s) sensor position, AED pad connector sensor position, Shock button sensor position, test duration (e.g., in minutes, seconds and/or milliseconds), carriage return, and/or line feed.

In some embodiments, once a test is completed (e.g., as shown in FIG. 16, once a test mode is completed), a user removes the thumb drive 321 from the AED device 400 and provides the thumb drive 321 to an administrator/instructor. The administrator/instructor logs onto a website configured to read data present on the thumb drive 321 and instructs the website to download test performance data (e.g., presses a load USB button on the screen). This uploads test performance data to the server. Test performance data is identified by user identification (e.g., on the first line of data). The user identification number links the data to a specific student. The data are analyzed by the website and a pass/fail is generated by the website. In some embodiments, the administrator/instructor can view the score and/or is able to print out the test taker's certificate. In some embodiments, the website is configured to allow a test taker to log into the website and permit the test taker to print their certificate (e.g., if they loose the original). In some embodiments, test performance data (from one or a plurality of users) is stored on the server. In some embodiments, the server and/or website is configured to generate an email to a user (e.g., test taker) to notify the user that their certification (e.g., CPR certification, AED certification) is about to expire/is expired (e.g., as a reminder to get recertified).

A system of the invention can be configured to provide a trainee and/or test taker multiple scenarios (e.g., training and/or testing scenarios). For example, a system described herein can provide a user one, two, three, four, five, six, seven, eight, nine, ten or more different scenarios (e.g., scenarios (e.g., chosen by an administrator and/or trainee) with different sequences of events (e.g., in a CPR and/or AED training process). Scenarios can be chosen (e.g., by a user) for training and/or testing via a website (e.g., from which information regarding a scenario is downloaded onto and/or programmed onto a USB drive (e.g., thumb drive) by an instructor/administrator, trainee, and/or test taker).

In some embodiments, the scenario is one person CPR (e.g., CPR administered by a single person). In some embodiments, the scenario is two person CPR (e.g., CPR administered by two persons). In some embodiments, the scenario is for single adult CPR and/or AED training. In some embodiments, the scenario is for single child CPR and/or AED training. In some embodiments, the present invention provides a system (e.g., CPR and/or AED training and/or testing system) comprising adult training scenarios. In some embodiments, the present invention provides a system (e.g., CPR and/or AED training and/or testing system) comprising child training scenarios. In some embodiments, a video training portion accompanies training in CPR and/or AED usage. In some embodiments, the present invention provides a system (e.g., CPR and/or AED training and/or testing system) comprising a written test.

Thus, in some embodiments, the present invention provides a system (e.g., CPR and/or AED training and/or testing system) comprising a USB drive (e.g., thumb drive (e.g., that comprises a configuration file (e.g., comprising one or more bytes of information (e.g., that correlate with training and/or testing scenarios (e.g., language of voice prompts, scenario data, CPR protocol, etc.))))). In some embodiments, the AED reads the file present on the USB drive and biased on each byte select, for example, a language (e.g., English, Spanish, French, German, Italian, user scenario, CPR protocol and/or other device configuration (e.g., volume or the like).

Data (e.g., user performance data) can be transferred from the USB drive (e.g., via a WAN (e.g., the internet)) to a remote server. In some embodiments, the remote sever stores the data in a database on storage media. Thus, in some embodiments, the present invention provides systems and methods for objectively collecting and storing data (e.g., user performance data (e.g., compared to the subjective nature of a human trainer/administrator observing and grading a user's performance)). In some embodiments, objective data (e.g., written skills test and the hands on testing with a mannequin) collected and/or stored (e.g., via a system of the present invention) provides the ability to formulate statistics from a database (e.g., that stores user performance data from a plurality of subjects (e.g., all subjects that have trained and/or tested utilizing a system described herein)). In some embodiments, statistical data generated is utilized to observe/characterize trending in what people (e.g., certain classes of people (e.g., elderly, young, emergency responders, etc.) do wrong and/or correct the most. Thus, systems and methods of the present invention can be utilized to improve the writing of CPR/AED protocols.

In some embodiments, anyone with a connection the internet can access the database (e.g., providing they have the correct credentials (e.g., such as username password and access level)). In some embodiments, anyone with a connection to the server with the correct user credentials may access the server.

In some embodiments, the present invention provides a website that provides a user interface to allow the user to access data, train in CPR manage certifications, obtain reminders certification is due, print certifications, view trend data (e.g., pass fail trends, specific question pass fail trending, specific hands on pass fail trending) or other piece of information obtained by the systems and methods of the invention. A user can schedule training and testing times. The user interface on the website allows the user to set up the modes of operation of the AED/mannequin, load test file data onto the thumb drive, and/or load test file data from the thumb drive to the database. The website also has the ability to charge for tests, has a storefront for the reordering of supplies, and/or allows for ordering mannequins, AEDs and/or components and/or accessories associated with each.

The systems and methods of the present invention may train, test and/or evaluate/certify a user's ability to learn and/or execute a number of skills and/or procedures including, but not limited to, placement of a tube (e.g., tracheal tube) into an airway (e.g., tracheal airway), cardiopulmonary resuscitation (CPR) ventilation, attachment of electrocardiogram (EKG) leads (e.g., EKG monitor sensing patches), reading of EKG data, blood pressure reading and/or attachment of pulse oximeter finger cuff attachment (e.g., of sensing patches), correct placement and/or use of an automatic external defibrillator (AED), and/or other skills and/or procedures related to medical care of a subject.

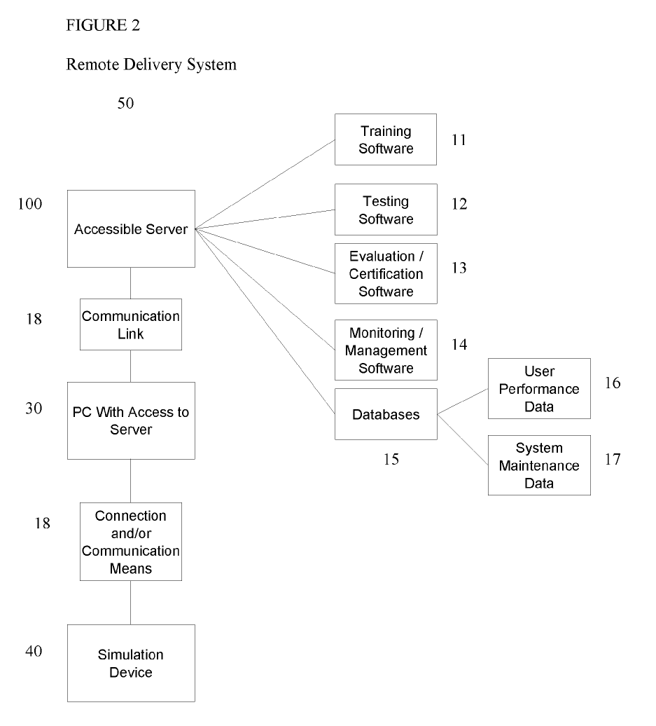

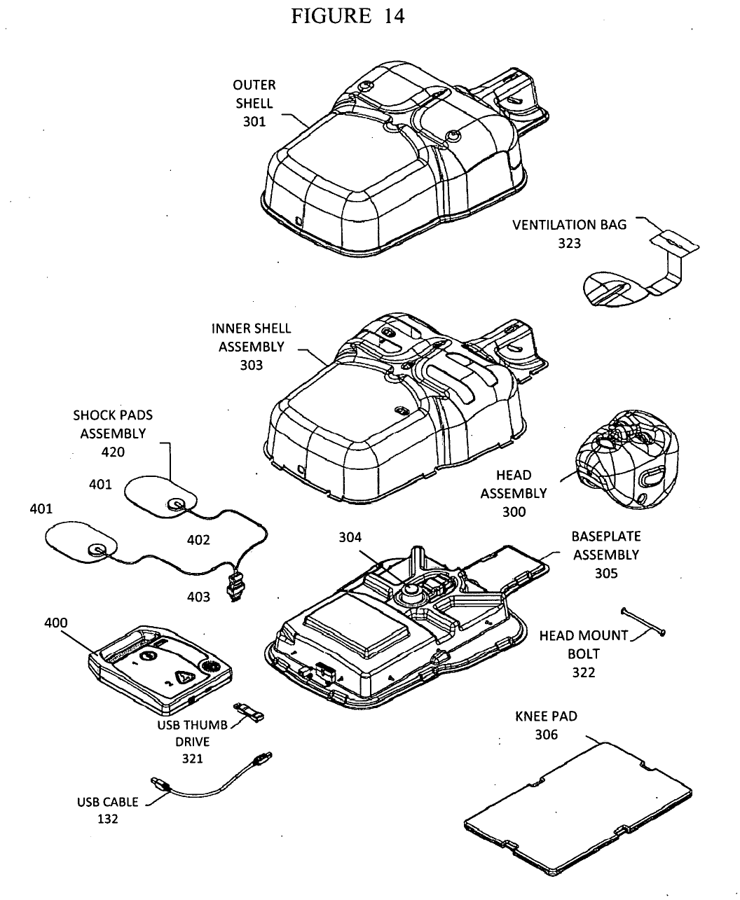

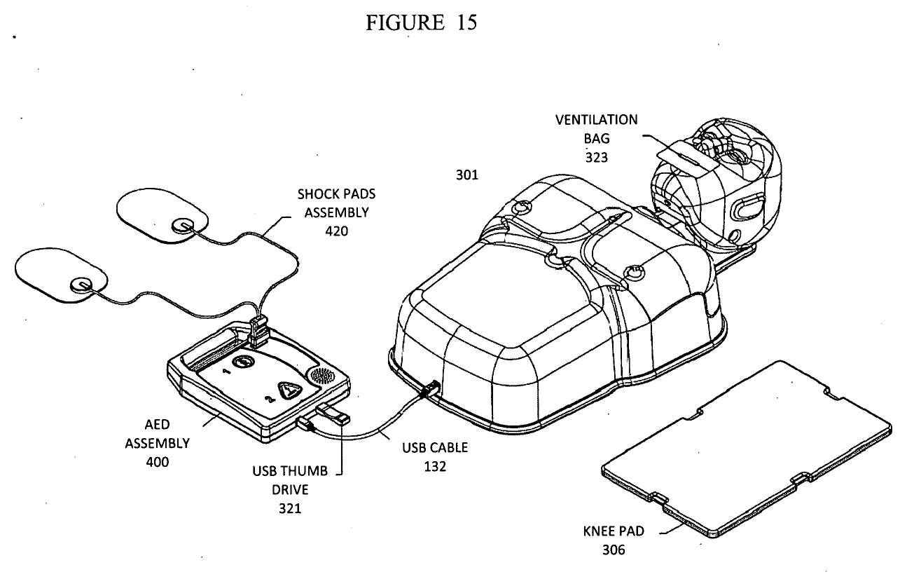

FIG. 2 describes one embodiment of remote delivery of software-based training 11, testing 12 and/or evaluation/certification software 13 (e.g., for a skill and/or procedure described herein) by an accessible server 100 (e.g., Web server) to a PC 30 connected to a simulation device 40. In some embodiments, a communication link 18 connects a server 100 to a computer 30 connected to and/or in communication with a simulation device 40. In some embodiments, as described herein, instead of connecting a server 100 to a computer 30, a communication link 18 connects a server 100 to data (e.g., user performance data, testing scenario data, mannequin and/or AED device 400 status data, etc.) present on a USB drive 321 (e.g., as shown in FIGS. 14 and 15). The present invention is not limited to any particular type of communication link 18, indeed a variety of communication links 18 are described herein including, but not limited to, an information network link (e.g. the Internet). Thus, the present invention provides a user (e.g., a trainee) of the systems and methods described herein the ability to obtain structured, skill-based training, testing and certification on specific software applications (e.g., depicted as 11, 12, and 13, respectively, in FIG. 2) via an accessible server 100. Moreover, in some embodiments, a user of (e.g., a test administer or authority placed in charge of regulating training, testing and/or certification) the systems and methods described herein gains the ability to monitor and manage the entire system via access to monitoring/management software 14 as well as databases 15 for storing information (e.g., user performance data) located on the server 100.

As described herein, the present invention is not limited by the type of accessible server 100 utilized. In some embodiments, the accessible server 100 is a conventional computing system capable of servicing training, testing and/or certification requests made by one or a plurality of users. Additional examples of accessible servers 100 include, but are not limited to, enterprise servers, midrange servers, workstations, or personal computer (PC) servers. In some embodiments, the accessible server 100 has the ability to access training 11, testing 12 and/or certification 13 management software and databases 15 (e.g., available on a SAN) for remotely administering and/or downloading training 11, testing 12 and/or certification 13 software applications onto one or more USB drive 321 (e.g., thumb drive)) and/or one or more PCs in communication with the accessible server 100. Several functions performed by training 11, testing 12 and/or certification 13 software include, but are not limited to, authenticating a training, testing and/or certification request from a user (e.g., an authorized user), building a training, testing and/or certification module corresponding to the user's training, testing and/or certification request, returning the training, testing and/or certification module to the USB drive 321 and/or PC with access to server 30, receiving, archiving and/or processing user training data 16 (e.g., user performance data (e.g., located on the USB drive 321) and/or system maintenance data 17 and/or certification information, as well as the ability to notify a certification authority (e.g., via authorized access to the data granted to the authority or via delivery of the data to the authority (e.g., at scheduled times (e.g., via electronic mail)) when one or more of the users achieve certification on a software application. In some embodiments monitoring and/or management software 14 is structured to provide real-time notification to a user (e.g., a certification authority, or a user) as to the certification status and/or skill-level of a remote user of the training 11, testing 12 and/or certification 13 software.

In some embodiments, databases 15 include archived user data 16 (e.g., user performance (e.g., training, testing and/or certification) results) and system maintenance data 17 (e.g., simulation device activity (e.g., sensor) data) and other types of data (e.g., entry and/or access logs, user logs, etc.). In some embodiments, databases 15 enable a manufacturer (e.g., of an AED simulation device 400, a PC 30 and/or a simulation device 40 (e.g., mannequin)) to maintain up-to-date lists of users (e.g., certified operators) of various software applications on the AED simulation device 400 (e.g., present on firmware of the AED device 400), PC 30 and/or a simulation device 40 connected thereto distributed throughout the world. Thus, if features are added or changed regarding the software applications the entire global community of users (e.g., certified operators) can be automatically notified of the changes by the manufacturer by broadcasting/notifying the change (e.g., via a communication link 18). Additionally, server databases 15 enable the manufacturers to accurately track which of the various software applications are most popular with and useful to users worldwide.

In some embodiments, a remote delivery system 50 comprises skill-based training activities corresponding to software applications resident on an AED simulation device 400, PC 30 and/or a simulation device 40 connected thereto. In some embodiments, skill-based training activities enable a user to undergo simulated self-paced training on software applications resident on the server 10.

In some embodiments, an AED simulation device 400, PC 30 and/or a simulation device 40 connected thereto permits a user to assess the performance of a simulation device 40 (e.g., mannequin comprising one or more sensors) and its associated circuitry via the communication link 18. For example, in some embodiments, procedures involving simulation device 40 functioning, programming, and/or transmission of operational information feedback from the simulation device 40 are controlled and/or monitored by the accessible server 100. These functions can be checked by a user (e.g., an authorized administrator (e.g., that manages the various functions of the system 50)). As described herein, an administrator or other authorized individual can manage (e.g., archive, review, format, analyze, mine, format (e.g., for presentation)) information (e.g., collected from one or a plurality of sensors residing within a simulation device) using a system of the present invention.

In some embodiments, once a user has received certification on one of the skills tested by software applications described herein, the server 100 can (e.g., in real-time) notify the user as well as identify the global location of the user and the level of certification (e.g., including the type of software). For example, an organization and/or authority (e.g., a hospital, school, day care provider, retirement community or home, city, town, state, etc.) may require notification that a user of one or more of the devices or procedures simulated by a simulation device described herein working within the organization (e.g., a hospital employee, a teacher, a day care worker, a nurse, an EMT, etc.) has achieved certification (e.g., in a procedure (e.g., CPR, AED usage, etc. (e.g., using systems and methods described herein (e.g., specific software applications for training, testing and/or certifying the user))) before the user is authorized to use the procedure and/or device (e.g., on behalf of the organization (e.g., for legal and/or liability reasons)). In some embodiments, user certification notification can be delivered, in real-time to the authority and/or organization (e.g., via authorized access to a database 15 on the server 10 or via electronic mail (e.g., via a communication link 18)).

In some embodiments, systems and methods of the present invention allow an organization to train their staff (e.g., in CPR and/or AED usage (e.g., in an automated process (e.g., at one or a plurality of sites (e.g., utilizing a mannequin (e.g., described in FIG. 3 and/or FIG. 8) or an AED (e.g., described in FIGS. 9, 11-15)))). In some embodiments, a system of the present invention also comprises a camera (e.g., a digital camera) that is capable of recording a user (e.g., training, testing and/or executing certification using a system and/or methods described herein). Thus, the present invention provides the capability for a subject (e.g., administrator, medical director, etc.) to monitor a specific user's physical performance of one or more skills in addition to monitoring a user's performance as gathered as data points (e.g., by one or more sensors residing within a simulation device).

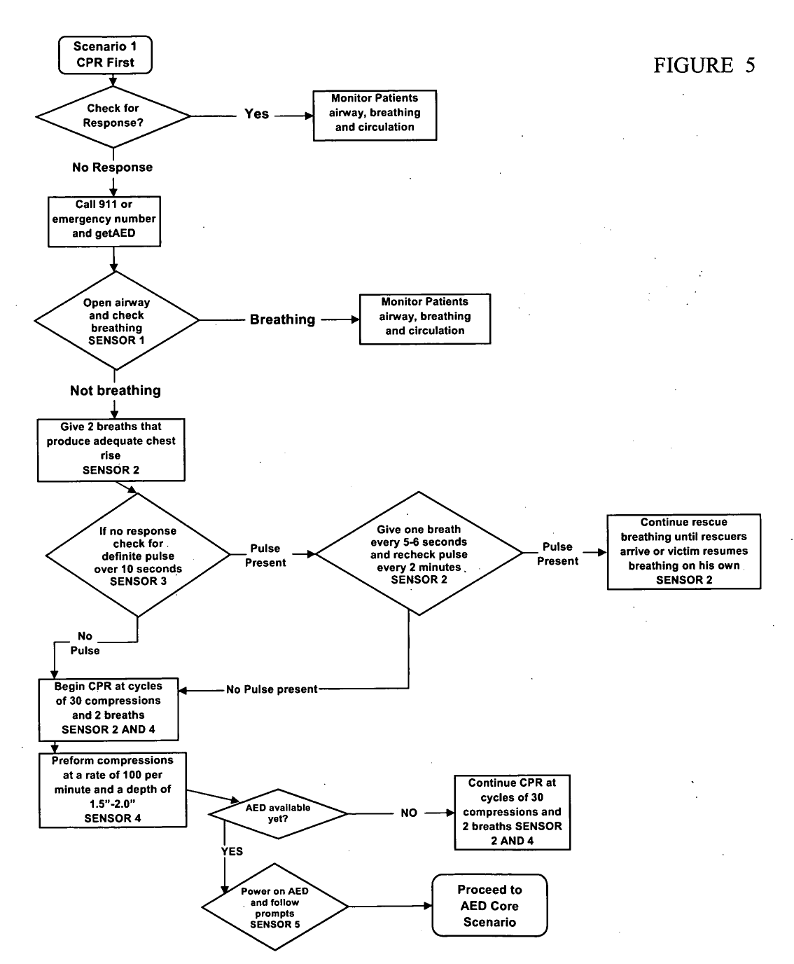

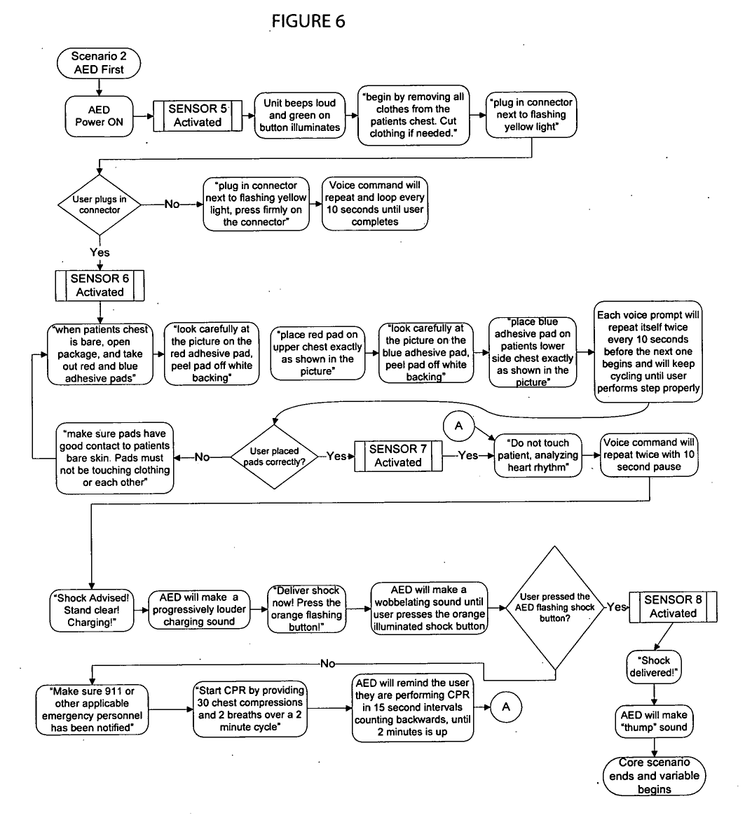

In some embodiments, the present invention provides a training and certification system (e.g., a CPR and/or AED online training and certification product) that uses video and/or interactive models to train (e.g., cognitively and/or emotionally) a user (e.g., online). In some embodiments, the present invention provides a training system comprising training, written test, and “hands-on” skill testing (e.g., via a mannequin (e.g., with sensors) and software (e.g., housed on a local PC or accessed from a server (e.g., internet accessible server))). In some embodiments, a system of the present invention utilizes any one of a number of approved (e.g., by a body and/or organization (e.g., American Heart Association, American Red Cross, and/or National Safety Council)) scenarios for assessing a users ability to perform CPR and/or utilize an AED. For example, in some embodiments, a system of the present invention utilizes testing (e.g., CPR and/or AED testing) logic as described in FIG. 5 and/or FIG. 6.

In some embodiments, a training and certification system comprises one or more of the following functions: training a user via an interactive video; administration and grading of a written test; providing hands-on skills training, practice and skills testing; execution of a certification process; production of credentials (e.g., certification credentials (e.g., wall certificate and/or wallet card)); organizational tools (e.g., available online from internet accessible server (e.g., that record user data (e.g., test and certification data (e.g., when a user becomes certified and/or when certification expires))) and alert and/or notification tools (e.g., that permit mailing (e.g., conventional and/or electronic mailing) of certification expiration renewal notices).

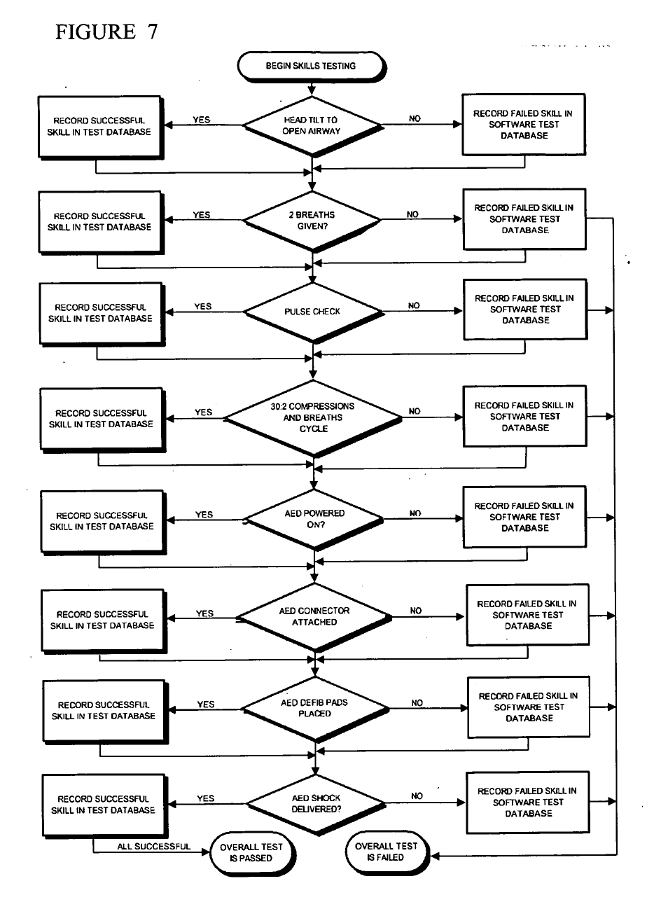

In some embodiments, a system of the present invention comprises a CPR and/or AED device training system (e.g., comprising one or more of a simulation device (e.g., a mannequin (e.g., with sensors that communicate a user's performance of required skills to perform cardio pulmonary resuscitation (CPR) and/or automated external defibrillation (AED))); an AED simulation device comprising firmware (e.g., that provides a skill based learning experience (e.g., described in FIGS. 5-7, 14-16)), communication means between the mannequin and AED simulation device, a computer (e.g., that allows communication with an AED simulation device and/or mannequin (e.g., a mannequin and/or AED system described herein (e.g., that are purchased and/or shipped together with a mannequin and/or AED system described herein))) with a server; a software program (e.g., firmware present on the AED device and/or available on an internet accessible server) that instructs and/or trains a user (e.g., regarding how to perform CPR and/or AED usage (e.g., operable in training, practicing and/or test taking mode)); and/or a website (e.g., housed on an internet accessible server) that serves as an online interface for an end user (e.g., an individual, organization or individual within an organization) to access software, systems and/or system components (e.g., online tools, databases, data analysis products, organizational tools, reporting or notification tools, and those available for purchase (e.g., mannequins, mannequin disposables (e.g., lung/airway bags and AED electrodes) tests, etc.)).

In some embodiments, the present invention provides a system (e.g., for training, testing and/or certifying one or more subjects (e.g., at the same time) in CPR and/or AED usage) comprising a mannequin. The present invention is not limited to the type of mannequin utilized. In some embodiments, a mannequin depicted in FIGS. 3, 14-15 and/or 17 is utilized. For example, a mannequin may comprise a plastic torso and/or head (e.g., capable or representing life-like positioning and/or status of a victim). A mannequin may also comprise one or more sensors. The present invention is not limited by the number, the type, nor the location of the sensor utilized. In some embodiments, a mannequin comprises a touch pressure sensor (e.g., in the neck (e.g., for recording pulse)); a magnetic prox sensor (e.g., in the mannequin head (e.g., to record head tilt and/or patient airway)) or other location (e.g., to record placement of AED defibrillation pads/electrodes). In some embodiments, a mannequin comprises one or a plurality of sensors (e.g., one or a plurality of each of the different types of sensors described herein). The present invention is not limited by the body part monitored nor the number of sensors utilized for such monitoring. In some embodiments, a mannequin comprises an arm (e.g., comprising a module representing blood pressure and/or a sensor for measuring the same). In some embodiments, a mannequin chest comprises a sensor for monitoring placement of one or a plurality of electrocardiogram (EKG) leads. In some embodiments, a mannequin comprises a finger (e.g., comprising a module representing oxygen saturation of the blood (e.g., to which a pulse oximeter can be attached). In some embodiments, a finger comprises a sensor (e.g., for monitoring placement of a pulse oximeter).

In some embodiments, the sensors are connected to a PC via a data acquisition module. The present invention is not limited by the type of data acquisition module. Indeed a variety of data acquisition modules can be utilized including, but not limited to a DLP-108-G module (DLP Design, Inc., Allen, Tex.) and other modules known in the art. In some embodiments, a data acquisition module has plurality of channels configured for acquisition of data. The present invention is not limited by the number of channels. In some embodiments, a data acquisition module has 4, 6, 8, 12, 16, or more (e.g., 24, 48 or more) channels.

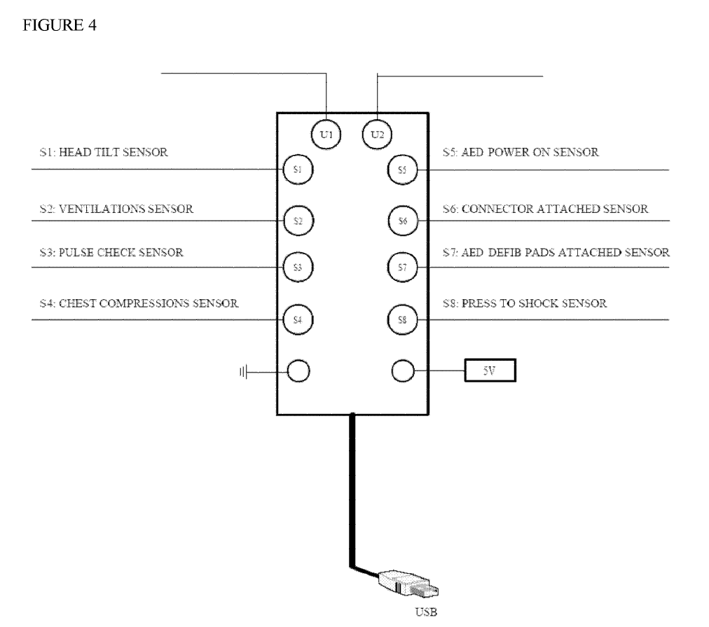

An example of a data acquisition module is shown in FIG. 4. In some embodiments, the data acquisition module comprises the ability to gather data from one or more sensors (e.g., sensors attached to a simulation device (e.g., a mannequin) described herein). In some embodiments, the data acquisition module comprises one or a plurality of channels (e.g., depicted in FIG. 4 as S1-S8) that are configured for acquisition of data (e.g., digital, analog, temperature, and other types of data). In some embodiments, the data acquisition module derives its power from a host USB port (e.g. from a user's PC or from a simulation device). In some embodiments, the data acquisition module is USB compatible (e.g., USB type 1, USB type 2, USB type 3, etc. compatible). In some embodiments, the data acquisition module comprises one or more applications including, but not limited to, data acquisition, industrial and/or process control, and process monitoring. In some embodiments, the data acquisition module is a 5-volt system, however, lower (e.g., from about 0 to about 5 volt) and higher (e.g., 5-10, 10-20, or higher) volt systems are also contemplated to be useful. In some embodiments, a data acquisition module provides data to a PC in the form of ASCII text (e.g., so that it is displayed and read using a simple terminal emulator). In some embodiments, a data acquisition module provides data to a PC in binary form (e.g., requiring a user's PC to possess an application (e.g., that processes and/or characterizes the data)).

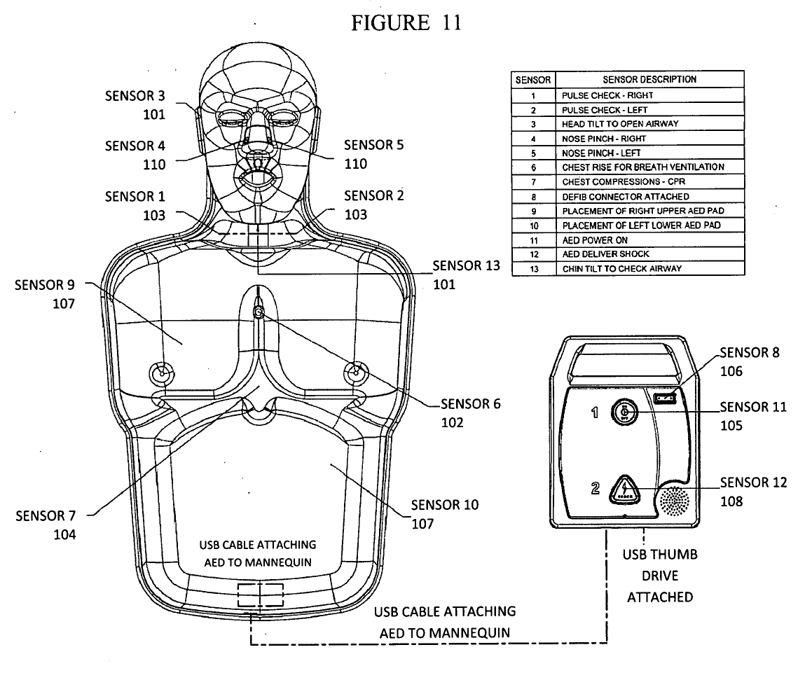

The present invention is not limited to any particular type of mannequin. In some embodiments, the present invention provides a mannequin assembly as shown in FIG. 8, FIG. 14 and/or FIG. 15. In some embodiments, the mannequin comprises an outer layer 301 (e.g., a soft, compressible outer layer (e.g., a foam outer layer)). The outer layer may comprise a single, or multiple pieces (e.g., fitted together to cover the entire top portion of torso portion of mannequin). In some embodiments, the mannequin comprises a chest plate 302. The present invention is not limited by the type of material used to make the chest plate. Indeed, a variety of materials may be utilized including, but not limited to, plastics, metals, synthetics, rubber, and other compounds. In some embodiments, the chest plate is configured for elevating and descending with regard to mannequin base 305. For example, the chest plate 302 may be attached to a base plate 305 and/or attached to a shell (e.g., plastic shell) 303 that is itself attached to base plate 305. The present invention is not limited by the type of attachment. Indeed, any attachment that permits movement of the plate 302 in an upward and downward motion in a vertical plane with regard to the base 305 is contemplated to be useful in the present invention. In some embodiments, the attachment is a securing channel between the chest plate 302 and shell 303 attached to a base 305. In some embodiments, a mannequin comprises a shell 303 (e.g., plastic shell (e.g., that is attached to a base plate 305)). The present invention is not limited by the type of material used to make the shell. Indeed, a variety of materials may be utilized including, but not limited to, plastics, metals, synthetics, rubber, and other compounds. In some embodiments, one or more other components (e.g., the head 300, chest plate 302, base plate 305, etc.) are attached to shell 303. As described herein, the present invention is not limited by the types of attachment. Indeed, a variety of attachments are contemplated to be useful including, but not limited to, removable attachments, sliding attachments, moving attachments, snap attachments, fixed attachments, swinging attachments, fit attachments, and other types of attachments that are well known in the art. In some embodiments, mannequin comprises a compression means 304 (e.g., the permits the rise and fall of chest plate 302 and/or outer shell layer 301 and/or inner shell assembly 303). The present invention is not limited by the type of compression means. For example, in some embodiments, compression means are provided by a compressible material that is placed in between base plate 305 and chest plate 302. In some embodiments, there is a void within the shell 303 for the compressible material 304 to fit. The present invention is not limited by the compressible material 304 utilized. Indeed, a variety of compressible materials may be used including, but not limited to, metal (e.g., metal springs), rubber or similar compounds, plastics, foams, etc. In some embodiments, the compressible material 304 is a foam. In some embodiments, the compressible material 304 is a spring. In some embodiments, the compressible material 304 is a combination of materials (e.g., spring loaded foam). In some embodiments, the compressible material 304 is attached to either base plate 305 and/or chest plate 302. In some embodiments, the compressible material 304 is attached to neither base plate 305 and/or chest plate 302, but rather is tightly fitted between the two. In some embodiments, the base plate 305 comprises one or more storage compartments (e.g., for removably storing other components used with the mannequin (e.g., AED unit components (AED base unit, AED pad pouch, etc.) power cables, knee pad, USB connections, etc.)). In some embodiments, mannequin comprises a knee pad 306. In some embodiments, the knee pad 306 fits into base plate 305 for storage. In some embodiments, mannequin comprises a head 300. In some embodiments, mannequin head 300 is attached to a base plate 305 and/or attached to shell 303 that is itself attached to base plate 305. The present invention is not limited by the type of attachment. Indeed, any attachment that permits tilting of the head 300 in one or more directions through the full range of motion that a human head tilts is contemplated to be useful in the present invention. In some embodiments, a mannequin comprises an attachment comprising track sliding that permits the head to tilt. In some embodiments, the mannequin comprises one or more sensors as described in detail herein. For example, in some embodiments, the present invention comprises chest rise and/or chest compression sensors (e.g., as shown in FIG. 11). Thus, the present invention provides a system that measures whether or not a user (e.g., trainee and/or test taker) has correct hand placement on the chest (e.g., during CPR (e.g., utilizing chest compression, chest rise, and/or cadence)). In some embodiments, one or more of the sensors comprise a Hamlin sensor (e.g., to detect head tilt and/or placement).

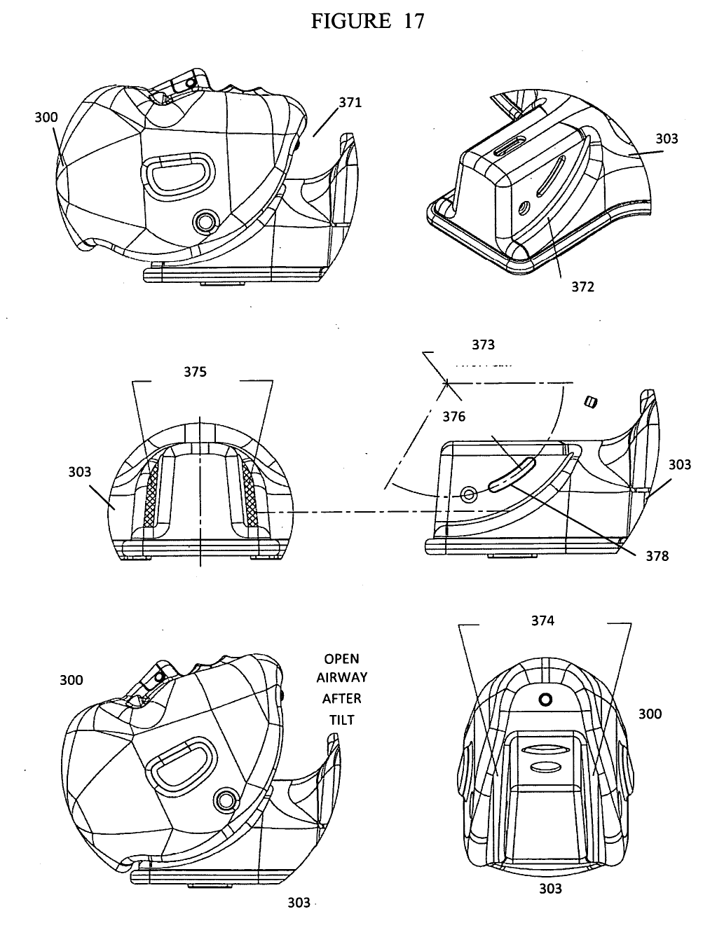

As shown in FIG. 17, in some embodiments, the inner shell assembly 303 comprises built in ramps 372 and a plurality (e.g., two) of rail surfaces 375 on the shell 303 for the head 300 to saddle and rotate on (e.g., to assist with a pendulum motion of head 300 pivot). Similarly, the head 300 comprises a surface area 374 on the head 300 which saddles to the rail.

A head mount bolt 322 (e.g., shown in FIG. 14) mounts the head 300 to the inner shell assembly 303. As shown in FIG. 17, The head mount bolt 322 rides on a channel 378 that is an arc. Pivoting the head 300 on this arc channel 378 simulates the movement of a human head in the motion of lifting the chin and tilting the head back to open the human's airway. The axis of rotational pivot point 373 and the centerline arc for rotational pendulum motion 376 are shown in FIG. 17. Utilizing this configuration, a mannequin of the present invention can comprise a closed airway 371 (e.g., prior to a trainee or test taker tilting of the head) and an open airway 377 (e.g., subsequent to a trainee or test taker tilting the head (e.g., to open the airway)).

In some embodiments, a system of the present invention comprises two or more mannequins. For example, is some embodiments, a system comprises a mannequin comprising means for connecting (e.g., directly or indirectly (e.g., via a data acquisition module) using wires, cables, wireless technology, etc.) to a PC and/or AED that comprises or that comprises access to software (e.g., firmware) for training, testing and/or certifying a user in one or more techniques (e.g., CPR and/or AED usage); as well as a mannequin that does not comprise such means for connecting to a PC and/or AED (e.g., a “practice mannequin”).

In some embodiments, AED firmware (e.g., USB firmware, AUDIO firmware, and/or main microprocessor firmware) can be updated via the USB thumb drive (e.g., the firmware architecture is “modular” and/or “open” allowing for upgrades (e.g., remotely updated (e.g., as CPR and/or AED protocols change)).

For example, an update file may be obtained via a website or other methods. In some embodiments, updates are run automatically via the recognition of one or more file(s) on the USB thumb drive. Thus, a device of the present invention can be upgraded and/or updated in the field (e.g., to accommodate changes in CPR specifications, to add and/or remove languages for the voice prompts of the device, to add and/or remove user scenarios, fix bugs, etc. in the field).

In some embodiments, a system of the present invention comprises software (e.g., firmware (e.g., that is installed and housed on a user's PC or AED device or that is accessible (e.g., via the internet) on a server) that operates (e.g., sends and receives information to and/or from) a mannequin and/or AED device (e.g., as shown in FIG. 16). In some embodiments, a mannequin is connected to a user's PC and/or AED device (e.g., via wireless or USB technology described herein). In some embodiments, software serves as a central dashboard for the user to perform various functions (e.g., host CPR and/or AED training videos and/or written CPR and/or AED tests (e.g., via ADOBE FLASH)). A PC and/or AED device housing software may or may not have an internet connection. If a connection is present, in some embodiments, the software records the users performance data and sends it (e.g., automatically) to a server (e.g., hosting a website (e.g., comprising training, testing and/or certification software) for a pass/fail analysis. In some embodiments, a thumb drive connected to an AED device records user performance data, and the data from the thumb drive is sent to a server when the thumb drive is connected to a PC connected to the internet. In some embodiments, if the PC has no internet connection, software stores the test information on the PC and/or thumb drive and uploads it later once an internet connection is established. In some embodiments, software is configured to provide a user with visual and/or voice information (e.g., confirmation) when a user is learning and/or practicing skills (e.g., prior to taking a test).

In some embodiments, a system of the present invention comprises a personal computer (e.g., shipped together with other components of the present invention). The present invention is not limited by the type of computer. Indeed, a variety of computers are contemplated to be a useful component of the present invention including, but not limited to, desktop, laptop, handheld, tablet, and other types of computers. Indeed, any computer comprising a processor and memory and/or memory device (e.g., for reading computer readable mediums) can be utilized with the present invention (e.g., an AED comprising a microcontroller, firmware and a USB drive/controller for connecting a USB drive). In some embodiments, a computer is shipped from company together with other components of a system of the present invention. In some embodiments, instead of an entire computer being shipped with a system of the present invention, only a component of a computer (e.g., a hard drive) is shipped with a system of the present invention. In some embodiments, a computer comprises a touch-screen interface (e.g., for a user to access, use, and enter commands regarding components of a system of the present invention). In some embodiments, a system of the present invention comprises barcode technology (e.g., a bar code reader (e.g., to read a barcode present on a user's identification and/or authorization document)). As used herein the term “processor” refers to a device that is able to read a program from a computer memory (e.g., ROM or other computer memory) and perform a set of steps according to the program. Processor may include non-algorithmic signal processing components (e.g., for analog signal processing). As used herein, the terms “memory,” “memory device,” “computer memory,” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape. As used herein, the term “computer readable medium” refers to any device or system for storing and providing information (e.g., data and instructions) to a computer processor. Examples of computer readable media include, but are not limited to, DVDs, CDs, hard disk drives, magnetic tape, flash memory, and servers (e.g., that provide information (e.g., stream media) over a network). In some embodiments, a computer of the present invention is able to receive, read, and/or provide multimedia information (e.g., to a user). As used herein the terms “multimedia information” and “media information” are used interchangeably to refer to information (e.g., digitized and analog information) encoding or representing audio, video, and/or text. Multimedia information may further carry information not corresponding to audio or video. Multimedia information may be transmitted from one location or device to a second location or device by methods including, but not limited to, electrical, optical, and satellite transmission, and the like. In some embodiments, a computer of the present invention is remotely accessible (e.g., via the internet or other type of network). As used herein, the term “internet” refers to any collection of networks using standard protocols. For example, the term includes a collection of interconnected (public and/or private) networks that are linked together by a set of standard protocols (such as TCP/IP, HTTP, and FTP) to form a global, distributed network. While this term is intended to refer to what is now commonly known as the Internet, it is also intended to encompass variations that may be made in the future, including changes and/or additions to existing standard protocols or integration with other media (e.g., television, radio, etc). The term is also intended to encompass non-public networks such as private (e.g., corporate) intranets. In some embodiments, a computer of the present invention comprises a security protocol. As used herein the term “security protocol” refers to an electronic security system (e.g., hardware and/or software) to limit access to processor, memory, etc. to specific users authorized to access the processor. For example, a security protocol may comprise a software program that locks out one or more functions of a processor until an appropriate password is entered. In some embodiments, a computer of the present invention comprises a resource manager. As used herein the term “resource manager” refers to a system that optimizes the performance of a processor or another system. For example a resource manager may be configured to execute one or more tasks including, but not limited to, monitor the performance of a processor or software application and manage data and processor allocation, perform component failure recoveries, optimize the receipt and transmission of data, and the like. In some embodiments, the resource manager comprises a software program provided on a computer system of the present invention. In some embodiments, a computer of the present invention is in electronic communication with one or more components (e.g., a mannequin, AED device, a website and/or server) of a system of the present invention. As used herein the term “in electronic communication” refers to electrical devices (e.g., computers, processors, communications equipment) that are configured to communicate with one another through direct or indirect signaling. For example, a conference bridge that is connected to a processor through a cable or wire, such that information can pass between the conference bridge and the processor, are in electronic communication with one another. Likewise, a computer configured to transmit (e.g., through cables, wires, wireless technology, infrared signals, telephone lines, etc) information to another computer or device, is in electronic communication with the other computer or device.

In some embodiments, the present invention also provides a website (e.g., housed on an internet accessible server) that interfaces with a system (e.g., comprising a mannequin, a user's PC housing software and/or AED device comprising a microcontroller and firmware, etc.) of the present invention. In some embodiments, a user will have and/or require a user login. In some embodiments, when the user is an organization (e.g., a school, day care facility, business, town, city, etc.), the organization will have and/or require an organizational login (e.g., coordinator's login). In some embodiments, the website will allow and/or require a user to login to view and/or take online training (e.g., provided as a FLASH video). In some embodiments, the website will allow and/or require a user to login to view and/or take written tests (e.g., provided as a FLASH video). In some embodiments, the website receives data (e.g., data files) from a user (e.g., user's performance data) and provides real-time (e.g., instant) analysis (e.g., pass/fail results (e.g., via algorithms present on the server housing the website or housed within the website located on the server)). A system of the present invention is able to acquire and store data/information (e.g., user performance data and/or test results) in a secure and confidential manner (e.g., in accordance with Health Insurance Portability and Accountability Act (HIPAA), Joint Commission on Accreditation of Healthcare Organizations (JCAHO), or other regulatory body rule or regulation). A website of the present invention can generate on-demand printing of certification (e.g., wall and or wallet card certificates). In some embodiments, the website comprises the ability to provide a user, an organization or a person within the organization (e.g., the organization's coordinator) a set of tools (e.g., including, but not limited to, personnel databases, expired certification reminders, follow-up letters, the ability to customize a written test from a pool of pre-written questions and answers and/or the ability to compose unique questions, scenario customization (e.g., CPR and/or AED scenarios (e.g., with different patient conditions, voice prompts, and/or outcomes)), purchasing tools (e.g., to order and/or re-order training and/or testing software, mannequins, mannequin consumables (e.g., lung/airway bags, AED electrodes, etc.), cross contamination barriers, educational material or other types of related material (e.g., that can be custom branded (e.g., with an organization's name)))).

Accordingly, in some embodiments, the present invention provides systems and methods of providing company products to a party outside of the company, for example, a system and method for providing a customer or a product distributor a product of the company such as a system and/or components thereof (e.g., software, mannequin, mannequin disposables, etc.) for CPR and/or AED training and testing. For example, in some embodiments the present invention provides a product management system. In some embodiments, the product management system comprises a company with intra-company organization that may comprise departments in a single building or in multiple building, a computer program (e.g., representing a department) and/or suite of programs (e.g., housed on one or more computers (e.g., a PC and/or a server located on a network)), a group of employees, a computer I/O device (e.g., a printer or fax machine), and/or a third party entity (e.g., a company) that is otherwise unaffiliated with the company.

In some embodiments, the company receives input (e.g., in the form of an order, information, or materials and parts) from a party outside of the company (e.g., a distributor and/or customer) to an order department; and provides output (e.g., in the form of a product delivered from shipping department (e.g., to a distributor and/or customer). In some embodiments, the product management system is organized to optimize receipt of orders and delivery (e.g., in a cost efficient manner) of products (e.g., a system and/or components thereof of the present invention) to a party outside of the company; and to obtain payment for such product from the party.

In some embodiments, the company comprises manufacturing and administration. Systems and components thereof of the present invention can be produced in manufacturing and/or by a third party, and can be stored separately therein such as in device storage and/or other component storage (e.g., product storage) and/or can be further assembled and stored (e.g., in device and/or product storage). Materials and parts (e.g., for assembly of systems and/or components thereof of the present invention) can be provided to company from an outside source (e.g., a third party) and/or materials and parts can be prepared in company (e.g., and used to produce devices and other components (e.g., that can in turn be assembled and sold as a product)). Manufacturing may also include a shipping department, which, upon receiving input as to an order, can ship a product to a party outside the company (e.g., after obtaining the product(s) to be shipped from device storage and/or other component storage and/or product storage).

In some embodiments, product storage can store instructions (e.g., for assembly and usage of a system and/or components thereof of the present invention). Upon receiving input from order department (e.g., that customer has ordered a product (e.g., system and/or component thereof of the present invention)) shipping department can obtain from product storage the product for shipping, and can further obtain such instructions in a written form to include with the product, and ship the product and instructions to customer (e.g., while concurrently and/or subsequently providing input to billing department (e.g., that the product was shipped)). In an alternative embodiments, the shipping department can obtain from product storage and/or other storage (e.g., electronic storage (e.g., on a server and/or database)) the product for shipping, and can provide the instructions and/or product to customer in an electronic form (e.g., by accessing a database in company (e.g., that contains the product and/or instructions) and transmitting (e.g., electronically transmitting) the product and/or instructions to customer (e.g., via the internet).

In some embodiments, administration includes order department (e.g., that receives input in the form of an order for a product from customer and/or distributor). Order department can then provide output in the form of instructions to shipping department to fill the order (i.e., to forward products as requested to customer or distributor). In some embodiments, the shipping department, in addition to filling an order, can also provide information/data to billing department in the form of confirmation of the products that have been shipped. In some embodiments, billing department provides output in the form of a bill to customer and/or distributor as appropriate, and can further receive input that the bill has been paid, or, if no such input is received, can further provide output to customer and/or distributor that payment has yet to be received (e.g., is delinquent). In some embodiments, other component of company may include customer service department (e.g., that can receive input from customer and/or provide output in the form of feedback or information to customer and/or can receive input or provide output to any other component of company). For example, customer service department can receive input from customer indicating that an ordered product was not received, wherein customer service department can provide output to shipping department and/or order department and/or billing department regarding the missing product (e.g. thereby providing means to assure customer satisfaction). Customer service department may also receive input from customer in the form of requested technical information, for example, for confirming that systems and methods of the invention can be applied to the particular need of customer, and can provide output to customer in the form of a response to the requested technical information.

Thus, in some embodiments, components of company are suitably configured to communicate with each other to facilitate the transfer of materials and parts, devices, other components, products, and information within and outside of company. For example, a physical path can be utilized to transfer products from product storage to shipping department upon receiving suitable input from order department. Order department, in comparison, can be linked electronically with other components within company, for example, by a communication network (e.g., a network (e.g. internet and/or intranet)), and can be further configured to receive input, for example, from customer by a telephone network, by mail or other carrier service, or via the internet. For electronic input and/or output, a direct electronic link (e.g., a T1 line or a direct wireless connection) may also be established (e.g., within the company and/or with a distributor).

In some embodiments, a product management system further comprises one or more data collection systems (e.g., including, but not limited to, a customer data collection system that can be realized as a personal computer, a computer network, a personal digital assistant (PDA), an audio recording medium, a document in which written entries are made, any suitable device capable of receiving data, and/or any combination of the same). Data collection systems can be used to gather data associated with a customer and/or distributor (e.g., including, but not limited to, a customer's shipping address and billing address, as well as more specific information such as the customer's ordering history and payment history (e.g., such data being useful, for example, to determine that a customer has made sufficient purchases to qualify for a discount on one or more future purchases)). In some embodiments, the one or more data collection systems collect data about user performance data (e.g., statistics related to test performance (e.g., of a certain group of users (e.g., type of user, location of user, etc.))).

In some embodiments, company can utilize a number of software applications to provide components of company with information and/or to provide a party outside of company access to one or more components of company (e.g., access to order department and/or customer service department and/or data collection/storage). Such software applications may comprise a communication network such as the Internet, a local area network, or an intranet. For example, in an internet-based application, customer can access a suitable website and/or a web server that cooperates with order department such that customer can provide input in the form of an order to order department. In response, order department can communicate with customer to confirm that the order has been received, and can further communicate with shipping department, providing input that products of the invention (e.g., mannequin, mannequin components, CPR and/or AED training and/or testing material/software, etc.) should be shipped to customer. Thus, in this manner, the business of company can proceed in an efficient manner.

Thus, in some embodiments, in a networked arrangement, various subcomponents of company (e.g., storage, billing department and shipping department) can communicate with one another by way of respective computer systems. As used herein, the term “computer system” refers to general purpose computer systems such as network servers, laptop systems, desktop systems, handheld systems, personal digital assistants, computing kiosks, and the like. Similarly, in accordance with known techniques, distributor can access a website maintained by company after establishing an online connection to the network, particularly to order department, and can provide input in the form of an order. If desired, a hard copy of an order placed with order department can be printed from the web browser application resident at distributor.

In some embodiments, various software modules associated with the implementation of the present invention can be suitably loaded into the computer systems resident at company and/or any party outside of company as desired, or the software (e.g., source code) can be stored on a computer-readable medium such as a floppy disk, magnetic tape, or an optical disk. In an online implementation, a server and website maintained by company can be configured to provide software downloads to remote users such as users, distributors, materials and parts suppliers, and the like. When implemented in software, various components of the present invention (e.g., training, testing, certification, user profiling, ordering, shipping, etc.) of the present invention are carried out by code segments and instructions associated with the various process tasks described herein.

In some embodiments, the present invention provides methods for providing various aspects of a product (e.g., systems and/or components thereof of the invention), as well as information regarding various aspects of a system of the present invention (e.g., user performance data, usage statistics, etc.), to parties (e.g., customers/users, distributors, third parties (e.g., accrediting organizations (e.g., American Heart Association, American Red Cross, National Safety Council, etc.)) etc). Thus, the present invention provides methods for selling systems, system components, devices, products, methods, and information of the present invention to such parties, as well as providing methods related to sales, including customer support, billing, product inventory, management within the company, etc. Examples of such methods include, but are not limited to, wherein materials and parts are acquired from a source outside of company (e.g., a supplier) and used to prepare components/devices used in preparing a system and/or composition or practicing a method of the invention (e.g., mannequins, mannequin disposables, software, AED device simulator (e.g., that can be maintained as inventory in product storage)).

It should be recognized that systems and/or components thereof can be sold directly to a customer and/or distributor, or can be combined with one or more other components and/or devices, and sold to a customer and/or distributor as the combined product (e.g., mannequin and computer system (e.g., housing software) sold together as a combined product). Other components (e.g., materials and parts) may be obtained from a source outside of company or can be prepared within company. As such, the term “product” is used generally herein to refer an item sent to a party outside of the company (a customer, a distributor, etc.) and includes items such as systems described herein or components thereof, which can be sent to a party alone or as a component of a system.

In some embodiments, a product is removed from product storage, for example, by shipping department, and sent to a requesting party such as customer or distributor. Typically, such shipping occurs in response to the party placing an order, which is then processed (e.g., forwarded to appropriate party) within the organization and results in the ordered product being sent to the party. Data regarding shipment of the product to the party can be transmitted further within the organization, for example, from shipping department to billing department, which, in turn, can transmit a bill to the party, either with the product, or at a time after the product has been sent. Further, a bill can be sent in instances where the party has not paid for the product shipped within a certain period of time (e.g., within 30 days, within 45 days, within 60 days, within 90 days, within 120 days, within from 30 days to 120 days, within from 45 days to 120 days, within from 60 days to 120 days, within from 90 days to 120 days, within from 30 days to 90 days, within from 30 days to 60 days, within from 30 days to 45 days, within from 60 days to 90 days, etc.). In some embodiments, billing department is responsible for processing payment(s) made by the party. It will be recognized that variations from the exemplified method can be utilized; for example, customer service department can receive an order from a party, and transmit the order to shipping department, thus serving the functions that can be carried out by order department and the customer service department.

The present invention also provides methods of providing technical service to parties using a system and/or components thereof of the invention. While such a function may be performed by individuals involved in product research and development, inquiries related to technical service can generally be handled, routed, and/or directed by an administrative department of the organization (e.g., customer service department). Technical service (e.g., solving problems related to use of the product or individual components of the product) communications may require exchange of information between a user (e.g., customer) and customer service department.

As mentioned above, any number of variations of a process of providing products (e.g., a system and/or components thereof described herein) and/or services to a user are possible and within the scope of the invention. Accordingly, the invention includes methods (e.g., business methods) that involve (1) the production of products (e.g., a system and/or components thereof described herein); (2) receiving orders for these products; (3) sending the products to parties placing such orders; (4) sending bills to parties obliged to pay for products sent to such; and/or (5) receiving payment for products sent to parties. For example, methods are provided that comprise two or more of the following steps: (a) obtaining parts, materials, and/or components from a supplier; (b) preparing one or more first products (e.g., one or more components of system described herein (e.g., mannequins, AED devices, computer, software, instructions, etc.) described herein)); (c) storing the one or more first products of step (b); (d) combining the one or more first products of step (b) with one or more other components to form one or more second products (e.g., a system described herein); (e) storing the one or more first products of step (b) or one or more second products of step (d); (f) obtaining an order for first product of step (b) or a second product of step (d); (g) shipping either the first product of step (b) or the second product of step (d) to the party that placed the order of step (f); (h) tracking data regarding the amount of money owed by the party to which the product is shipped in step (g); (i) sending a bill to the party to which the product is shipped in step (g); (j) obtaining payment for the product shipped in step (g) (generally, but not necessarily, the payment is made by the party to which the product was shipped in step (g)); and (k) exchanging technical information between the organization and a party in possession of a product shipped in step (d) (typically, the party to which the product was shipped in step (g)).

Mannequins used in systems and methods of the invention may be designed so that they do not communicate with a computer unless the mannequin and/or computer comprises means (e.g., source code, software, communication technology, etc.) of communicating with each other. An advantage of this is that purchasers may have to purchase software for their particular application. An advantage of this is that a customer may be able to customize a particular system to their needs (e.g., purchase software that permits communication between a single mannequin and a single computer, or purchase software that permits communication between a plurality of mannequins (e.g., 2 or more, 5 or more, 10 or more, 20 or more, 50 or more, 75 or more, 100 or more) and a single computer.

The present invention also provides a system and method for providing information as to availability of a product (e.g., a system or component thereof) to parties having potential interest in the availability of the systems and components thereof described herein. Such a method of the invention, which encompasses a method of advertising to the general or a specified public, the availability of the product (e.g., comprising a system and/or component thereof of the present invention) can be performed, for example, by transmitting product description data to an output source, for example, an advertiser; further transmitting to the output source instructions to publish the product information data in media accessible to the potential interested parties; and detecting publication of the data in the media, thereby providing information as to availability of the product to parties having potential interest in the availability of the product. It is also contemplated that this information can be transmitted to parties that have shown or that may be likely to show interest in acquiring a financial interest in the company.

Accordingly, the present invention provides methods for advertising and/or marketing systems and/or components thereof, products, and/or methods of the invention, such methods providing the advantage of inducing and/or increasing the sales of such systems, products, and/or methods. For example, advertising and/or marketing systems and methods of the invention include those in which technical specifications and/or descriptions of systems and/or products; methods of using the systems or components thereof, and/or products; and/or instructions for practicing the methods and/or using the systems and/or products are presented to potential interested parties, particularly potential purchasers of the product such as customers, distributors, and the like. In particular embodiments, the advertising and/or marketing methods involve presenting such information in a tangible form or in an intangible to the potential interested parties. As disclosed herein and well known in the art, the term “intangible form” means a form that cannot be physically handled and includes, for example, electronic media (e.g., e-mail, internet web pages, etc.), broadcasts (e.g., television, radio, etc.), and direct contacts (e.g., telephone calls between individuals, between automated machines and individuals, between machines, etc.); whereas the term “tangible form” means a form that can be physically handled.

The invention further provides methods associated with the design of custom systems and products. These methods include, for example, (1) taking an order from a customer for a system with specific subcomponents and/or means for operating the same, (2) preparation of the system with specific subcomponents and/or means for operating the same, (3) and providing (e.g., shipping) the system of (b) to the customer. Additionally, in particular embodiments, the customer may be billed for the system with the bill either being sent to the customer along with the system or sent separately.

In some embodiments, the present invention provides an information providing management system as encompassed within the present invention. In some embodiments, the information providing management system may comprise an intra-company organization that may comprise departments in a single building or in different buildings, a computer program or suite of programs maintained by one or more computers, a group of employees, a computer I/O device such as a printer or fax machine, a third party entity or company that is otherwise unaffiliated with the company, or the like.

The information providing management system can be exemplified by company, which makes, purchases, or otherwise makes available systems and methods that alone, or in combination, provide products, for example, systems and/or system components of the present invention, that company wishes to sell to interested parties. To this end, product descriptions are made, providing information that would lead potential users to believe that products can be useful to user. In order to effect transfer of product descriptions to the potential users, product descriptions may be provided to advertising agency, which can be an entity separate from company, or to advertising department, which can be an entity related to company, for example, a subsidiary. Based on the product descriptions, advertisement is generated and is provided to media accessible to potential purchasers of products, whom may then contact company to purchase products.

For example, product descriptions can be in a tangible form such as written descriptions, which can be delivered (e.g., mailed, couriered, etc) to advertising agency and/or advertising department, or can be in an intangible form such as entered into and stored in a database (e.g., on a computer, in an electronic media, etc.) and transmitted to advertising agency and/or advertising department over a telephone line, T1 line, wireless network, or the like. Similarly, advertisement can be a tangible or intangible form such that it conveniently and effectively can be provided to potential parties of interest (e.g., potential purchasers of product). For example, advertisement can be provided in printed form as flyers (e.g., at a meeting or other congregation of potential interested parties) or as printed pages (or portions thereof) in magazines known to be read by the potential interested parties (e.g., trade magazines, journals, newspapers, etc.). In addition, or alternatively, advertisement can be provided in the form of directed mailing of computer media containing the advertisement (e.g., CDs, DVDs, floppy discs, etc.) or email (e.g., mail or e-mail that is sent only to selected parties, for example, parties known to members of an organization that includes or is likely to include potential users of products); of web pages (e.g., on a website provided by company, or having links to the company website); or of pop-up or pop-under ads on web pages known to be visited by potential purchaser of products, and the like. Potential purchasers of products, upon being apprised of the availability of the products (e.g., systems and system components thereof of the present invention), can then contact company and, if so desired, can order products from company.

We claim:

1. An interactive cardiac pulmonary resuscitation (CPR) and/or automated external defibrillator (AED) training and/or testing system comprising:
a) a AED simulation device, wherein said device comprises a microcontroller;
b) a mannequin, wherein said mannequin comprises a microcontroller; and
c) a bi-directional communication link between the AED simulation device microcontroller and the mannequin microcontroller.

2. The system of claim 1, wherein said AED simulation device comprises firmware.

3. The system of claim 2, wherein said firmware comprises universal serial bus (USB) firmware, audio firmware, and microprocessor firmware.

4. The system of claim 1, wherein said AED simulation device comprises a USB drive and/or controller.

5. The system of claim 4, wherein the USB drive is utilized to update firmware of said AED simulation device.

6. The system of claim 4, wherein testing and/or training data is stored on a USB drive connected to the AED simulation device.

7. The system of claim 6, wherein information is communicated between the AED simulation device microcontroller, the mannequin microcontroller, and the USB drive.

8. The system of claim 6, wherein said testing and/or training data comprises user performance data.

9. The system of claim 1, wherein said bi-directional communication link comprises a USB cable.

10. The system of claim 1, wherein said AED simulation device and said mannequin comprise one or more sensors.

11. The system of claim 10, wherein said one or more sensors are selected from the group consisting of a head tilt sensor, pulse sensor, compression sensor, ventilation sensor, location sensor and a charge sensor.

12. The system of claim 10, wherein said sensors comprise hall effect sensors, tactile switch sensors and/or magnetic proximity sensors.

13. The system of claim 12, wherein said sensors collect user performance data.

14. The system of claim 1, wherein said AED simulation device and said mannequin comprise one or more light-emitting diodes (LEDs).

15. The system of claim 14, wherein said one or more LEDs are located at one or more positions comprising a shock switch of the AED device, an on/off button of the AED device, location of chest rise and/or compression of the mannequin, location of AED pad placement on the mannequin, location of head tilt on the mannequin, location of pulse check on the mannequin, and location of nose pinch of the mannequin.

16. The system of claim 15, wherein firmware present in said AED simulation device utilizes a protocol that illuminates said one or more LEDs at a location where a user of the system needs to perform an action and extinguishes said one or more LEDs when a proper action has been completed.

17. The system of claim 1, wherein said mannequin comprises a translucent poly vinyl cover.

18. The system of claim 1, wherein said AED simulation device comprises a speaker.

19. The system of claim 18, wherein said speaker provides voice prompts to a user of the system.

20. The system of claim 19, wherein said voice prompts comprise instructions to a user training on the system in the performance of CPR and/or AED usage.

21. The system of claim 1, wherein said system comprises an internet accessible server.

22. The system of claim 21, wherein said server houses software for training, testing and/or certifying a user in CPR and/or AED usage.

23. The system of claim 21, wherein said server houses a database of user performance data.

24. The system of claim 23, wherein said database is utilized to generate performance statistics.

25. The system of claim 21, wherein said system comprises a website.

26. The system of claim 25, wherein the website provides a user interface that allows a user access to user performance data, certification information, or trending data.

27. An interactive cardiac pulmonary resuscitation (CPR) and/or automated external defibrillator (AED) training and/or testing system comprising:
a) an AED training unit assembly comprising a microcontroller;
b) a shock pad assembly comprising a pair of defibrillator pads, a pair a wires, and an attachment; and
c) a mannequin.

28. The system of claim 27, wherein said mannequin comprises a head and an inner shell assembly comprising built in ramps and a plurality of rail surfaces on the shell for the head to saddle and rotate upon, wherein a head mount bolt mounts the head to the inner shell assembly.

29. The system of claim 28, wherein the head mount bolt rides on a channel of the inner shell assembly that is an arc.

30. The system of claim 29, wherein pivoting the head on the arc channel simulates the movement of a human head in the motion of lifting the chin and tilting the head back to open a human's airway.
